(19) United States
(12) Patent Application Publication
(10) Pub. No.: US 2019/0084903 A1
(43) Pub. Date: Mar. 21, 2019

(54) CATALYTIC ETHENOLYSIS OF OPTIONALLY-FUNCTIONALIZED INTERNAL UNSATURATED OLEFINS

(71) Applicants: TOTAL RAFFINAGE CHIMIE, Courbevoie (FR); CENTRE NATIONAL DE LA RECHERCHE SCIENTIFIQUE (CNRS), Paris Cedex 16 (FR); UNIVERSITE CLAUDE BERNARD LYON, Villeurbanne (FR); ECOLE SUPERIEURE DE CHIME PHYSIQUE ELECTRONIQUE DE LYON, Villeurbanne (FR)

(72) Inventors: Mostafa TAOUFIK, Villeurbanne (FR); Régis GAUVIN, Lille (FR); Laurent DELEVOYE, Bourghelles (FR); Pascal ROUGE, Villeurbanne (FR); Kai Chung SZETO, Villeurbanne (FR); Yassine BOUHOUTE, Villeurbanne (FR); Henri STRUB, Pont Sainte Maxence (FR)

(73) Assignees: TOTAL RAFFINAGE CHIMIE, Courbevoie (FR); CENTRE NATIONAL DE LA RECHERCHE SCIENTIFIQUE (CNRS), Paris (FR); UNIVERSITE CLAUDE BERNARD LYON, Villeurbanne (FR); ECOLE SUPERIEURE DE CHIMIE PHYSIQUE ELECTRONIQUE DE LYON, Villeurbanne (FR)

(21) Appl. No.: 15/745,249

(22) PCT Filed: Jul. 18, 2016
(86) PCT No.: PCT/EP2016/067055

§ 371 Date: Jan. 16, 2018

(30) Foreign Application Priority Data
Jul. 17, 2015 (EP) 15306174.2

(51) Int. Cl. C07C 6/04; B01J 31/16; B01J 31/22; C07C 2/30; C07C 11/02; C08F 4/78; C08L 23/24

(52) U.S. Cl. CPC C07C 6/04; B01J 31/1608; B01J 31/2265; C07C 2/30; C07C 11/02; C08F 4/78; B01J 2531/66; C07C 2531/22; C07C 2531/34; C07C 2521/08; C07C 2521/04; B01J 2231/543; B01J 2531/64; C08L 23/24

(57) ABSTRACT

The disclosure relates to a process for obtaining alpha-olefins by heterogeneous catalytic ethenolysis of optionally-functionalized unsaturated, in particular mono-unsaturated, olefins. The disclosure also relates to new supported catalysts that can be used in the process and to a method for preparing the supported catalysts.

### CROSS-REFERENCE TO RELATED APPLICATIONS

This application is a National Phase Entry of International Patent Application No. PCT/EP2016/067055, filed on Jul. 18, 2016, which claims priority to European Patent Application Serial No. 15306174.2, filed on Jul. 17, 2015, both of which are incorporated by reference herein.

### TECHNICAL FIELD

The invention relates to a process for obtaining alpha-olefins by heterogeneous catalytic ethenolysis of optionally-functionalized internal unsaturated, in particular mono-unsaturated, olefins. The invention also relates to new supported catalysts that can be used in the process of the invention and to a method for preparing said supported catalysts.

### BACKGROUND

Major challenge in sustainable petrochemical industries is to replace the gradually depletion of fossil petroleum-derived raw materials with renewable feedstocks. In particular, valorization of downstream products originated from bio-refinery has gained traction in recent years. Unlike petrochemicals, oleochemicals are derived from renewable resources, have acceptable bio-degradability and CO₂ neutral. In addition, oleochemicals are attractive bio-refinery feedstocks due to their availability on large scale at reasonable prices and these products present multiple functional groups allowing further chemical modifications to valuable products.

Natural fats and oils (glyceryl esters of fatty acids) are readily available raw materials for oleochemical industry. About 14% of the world production of fats and oils (annual production 103 million tons) is used in the oleochemical industry as starting material. The most important are the long-chain vegetable oils (soybean, sunflower, rapeseed, etc.) which contain mainly unsaturated C18 oleic acids, and are important sources for the production of cosmetics, detergents, soaps, emulsifiers, polymer additives, etc. Generally, the extracted poly-ester oils are converted to monoester in order to simplify further chemical treatment with a high purity of the final products.

In fact, fatty acid monoesters are usually obtained from the transesterification of natural oils and fats with a lower alcohol, e.g., methanol, along with glycerol. More than 90% of all oleochemical reactions (conversion into fatty alcohols and fatty amines) of fatty acid esters is carried out at the carboxy function. However, transformations by reactions of the carbon-carbon double bond, such as hydrogenation, epoxidation, ozonolysis and dimerization, are becoming increasingly important industrially. Among the different chemical pathways to upgrade fatty acid monoesters (for example methyl oleate), olefin metathesis has emerged as a powerful tool to produce valuable products after redistribution of C—C double bonds.

A considerable share of industrial processes relies on catalysis, which plays a strategic role in the production of a wide range of chemicals. Among the reactions that have been carried out by catalysts, olefin metathesis occupies an important position, not only in the petrochemical but also in fine chemicals and oleochemical sectors. This atom-economical transformation consists in the exchange of alkylidene fragments between two olefins. This reaction is catalyzed by homogeneous or heterogeneous systems involving a transition metal (Mo, Ru, W or Re). The complete mechanism has been revealed by Hérisson and Chauvin in 1971. The key element of this mechanism is a metallocarbene specie that reacts with an olefin to form a metallacyclobutane. The former evolves to give a new olefin and a new metallocarbene.

The overall importance of olefin metathesis in organic synthesis has been globally highlighted in 2005 by awarding the Nobel price to Robert H. Grubbs, Richard R. Schrock and Yves Chauvin for their contribution to olefin metathesis development. This revolutionary reaction allows reducing the number of steps of certain process. Consequently, it becomes central in the development of green chemistry and more environmental friendly processes. There are two main types of metathesis reactions: self-metathesis of an olefin (functionalized or not) with itself; cross metathesis of an olefin (functionalized or not) with another olefin.

The self-metathesis of methyl esters such as methyl oleate (Scheme 1) gives to the formation of 9-octadecene (C18) and dimethyl-9-octadecene-1,18-dioate (C18):

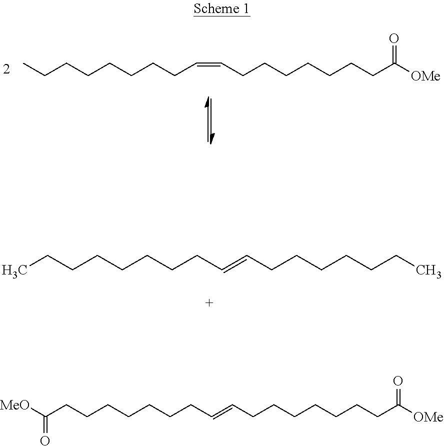

The formed products from self-metathesis have potential applications as biodiesel and production of polymers. The diester is particularly interesting for the production of biodegradable polyester after reaction with diol. Alternatively, the diester can also be converted into typical musk molecules (civetone) by Dieckmann condensation followed by hydrolysis and decarboxylation, frequently used in perfumery industry.

Metathesis of fatty acid monoester has been demonstrated by Boelhouwer et al. already in 1972. Self-metathesis of methyl oleate has been performed in the presence of the homogenous catalyst formed by WCl₆ and alkylating agent SnMe₄. At 110° C., this catalytic system offered a turn-over number (TON) of 38 after 2 hours. Improved activity can be obtained by W(O-2,6-C₆H₃X₂)₂Cl₄ (X═Cl,Ph) promoted by SnMe₄.

Self-metathesis of methyl oleate can also be directly performed without co-catalyst in homogenous system by tungsten and molybdenum imido complexes (M(═CHCMe₃)(═NC6H3-iPr2-2,6-)[OCMe(CF₃)₂]₂; TON=250 when M=W). Note that these complexes contain already a carbene moiety that can undergo metathesis reaction. Enhanced activity can be obtained when supporting the organometallic complexes on conventional supports by surface organometallic chemistry. The supported species avoid bimolecular decomposition which is the main deactivation route for these systems.

Other active transition metal in olefin metathesis is rhenium. The simple Re₂O₇/Al₂O₃ system converts unsaturated esters by metathesis after alkylation by SnMe₄. Further optimization of the activity can be obtained by tuning the support (introducing doping agents, such as silicon or boron) and alkylating agents (such as SnBu₄, SnEt₄, GeBu₄, PbBu₄). Moreover, the development of MeReO₃ supported on alumina or silica-alumina allows catalytic self-metathesis of methyl oleate without alkylating agents. The active carbene specie is obtained by coordination of the oxo ligand with surface Lewis sites.

In contrast to the oxophilic early transition metals, the catalytic systems based on group 8 transition metals are more tolerant towards functionalized metathesis substrates. In particular, catalysts based on ruthenium have been largely explored for methyl oleate self-metathesis. For example, a TON of 440000 can be achieved by Grubb's 2^{nd} generation catalyst RuCl₂(═CHPh)(SIMes)(PCy₃). But this catalyst suffers from low selectivity, originated from isomerization reactions. In general, ruthenium complexes used for methyl oleate self-metathesis exhibit higher TON than the supported rhenium system. However, the immobilization of ruthenium catalysts for methyl oleate self-metathesis often exhibits significantly loss of activity. In US 2013/026312 document, Materia Inc has developed a new approach to immobilize Ru-based olefin metathesis catalysts by anchoring the ruthenium complexes on two different linkers (scheme 2). These catalysts have shown a high activity and stability in methyl oleate self-metathesis.

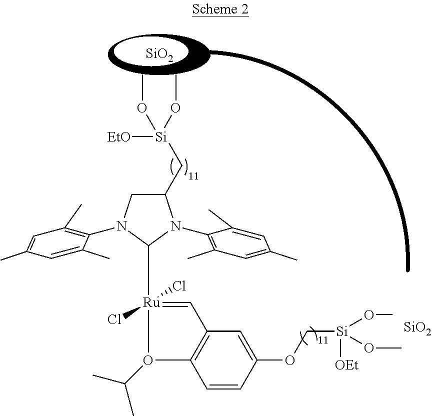

Another mode of reactivity with oleochemicals, “ethenolysis,” i.e. the olefin metathesis reaction with ethylene, is of particular interest because of the terminal olefin products that are formed. In particular, ethenolysis of methyl oleate gives 1-decene and methyl 9-decenoate. These molecules are potentially useful as an intermediate for surfactants, polymer additives, surface coatings, lubricants and other products. Excess of ethene can easily be applied (e.g., by using elevated ethene pressures) to suppress self-metathesis of the ester and to force the conversion to completion.

For efficient production of the diester of methyl oleate a two-step process can be considered. First, methyl oleate undergoes ethenolysis to dec-1-ene and methyl dec-9-enoate; high conversions can be obtained by using a high ethene pressure. After product separation, methyl dec-9-enoate undergoes self-metathesis to ethene and dimethyl octadec-9-enoate. Equilibrium can be shifted by continuous removal of ethylene. In this way more than 50% conversion can be obtained in both reaction steps, and there are no big problems in separating the reaction products. A problem is the deactivation of catalytic sites by the ester group resulting in reduced activities than those obtained for the metathesis of analogous simple olefins. Because of the potential industrial importance of this reaction, much effort has been devoted to the development of catalysts based on early transition metal (Mo, W and Re) able to conduct the cross-metathesis of unsaturated fatty acid esters with ethene. The most active homogeneous catalyst systems are the well-defined metal alkylidene complexes (exemplified in scheme 3) in its highest oxidation state. The high activity is also assisted by bulky electron-withdrawing ligands (aryloxides, fluoroalkoxides, imido); that prevent deactivation by dimerization and the co-ordination of the functional group to the metal atom.

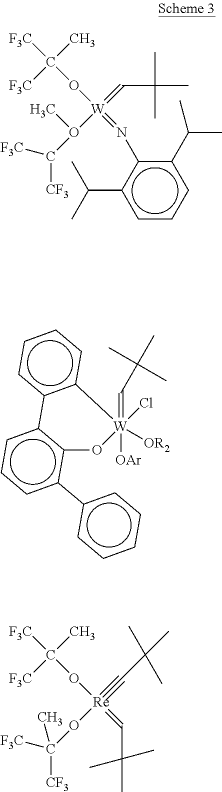

A highly active system is reported by Schrock et al. (R. R. Schrock, J. AM. CHEM. SOC. 2009, 131, 10840-10841) using molybdenum imido alkylidene complexes with bulky aryloxide groups (TON up to 4750) with a selectivity of >99% and yields up to 95% (Scheme 4). However, using tungstacyclobutane catalysts gives lower activity (TON=310) than the molybdenum catalysts, although the selectivity remains high. The difference in activity between both group 6 metals is explained either by the difficulty to release ethylene from a stable unsubstituted metallacyclobutane, or by coordination of the ester carbonyl group to physically bigger tungsten center compared to molybdenum.

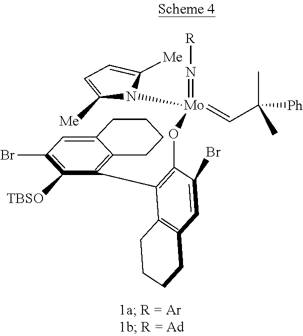

Alternatively, a simple mixing of WOCl₄ or WCl₆ with a suitable cocatalyst (an alkylating agent such as tetra-alkyl tin or silicon) catalyzes ethenolysis reaction of methyl oleate. These catalytic systems are cheap, commercially available and easier to handle than the alkylidene complexes. Applying bulky aryloxide ligands, such as W(OAr)₂Cl₄, allowing its manipulation under air, some catalytic systems have been developed in presence of cocatalyst that by alkylation give an alkylidene active site.

In heterogenous catalysis, Re₂O₇/Al₂O₃/Me₄Sn was the first catalyst found to be effective for the metathesis of olefinic esters. Different parameters have been studied in order to improve this system. A promising catalyst is based on doped support (with silicon and boron) along with SnBu₄ as promoter (TON=348). Although rhenium-based systems are only active for the metathesis of functionalized olefins when promoted with an alkyltin or alkyllead compound, the role of the latter is still not well understood. Reduction of the rhenium atom, modification of the active site (by addition of a tin ligand) and formation of the initiating metal-alkylidene species (via a double alkylation followed by an □-H-abstraction) have been postulated as promotion mechanisms.

The most studied catalytic system for ethenolysis of methyl oleate is based on ruthenium. The non-oxophilic nature provides an exceptional resistance towards hetero-atomic groups in the substrate. Maughon et al. in collaboration with Dow Chemical Company demonstratred the first generation Grubbs catalysts which give high TON 15 000 in ethenolysis of methyl oleate (Maughon, B. R. Organometallics 2004, 23, 2027-2047). It has been noted that the catalyst deactivates with the conversion, which is due to formation of α-olefins in the reaction.

Elevated TON (35000) has been obtained with the commercial catalyst from Materia Inc developed in collaboration with Grubbs (scheme 5, Grubbs, R. H. Organometallics 2008, 27, 563-566). According to an economic evaluation by Dow, ruthenium-based catalysts need to exceed a TON of 50000 in order to be economically viable. Before commercialization, several issues require to be addressed: increasing the active site (ruthenium carbene moiety) lifetime; decrease of the concentration of the produced terminal olefins during the reaction by continuous removal.

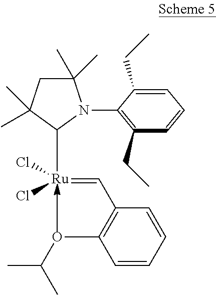

A common pathway for ruthenium catalyst deactivation is the facile decomposition of metallacyclobutane followed by a reduction of the metal initiated by ruthenium methylidene moiety. The latter specie is inevitably formed in the presence of terminal olefins. Moreover, the formed α-olefins will also undergo coordinating competition to the ruthenium center with the sterically hindered substrate (methyl oleate), and thus decrease the productivity. Hence, to increase the lifetime of the active ruthenium species and the activity, it is necessary to remove the α-olefins formed during the metathesis reaction, by for example reactive distillation or working under continuous flow. A chemical approach to avoid formation of ruthenium methylidene species is to apply an internal olefin in the cross metathesis reaction with methyl oleate. 2-butenes have already been used and have shown enhanced TON (440000) for the 2^{nd} generation Grubb's catalyst (Patel J., Chem. Commun. 2005, 5546-5547). However, the latter method requires a supplementary and difficult step that is the isomerization of internal olefins to terminal olefins, making this reaction less attractive for the industry. Therefore, ethenolysis of methyl oleate catalyzed by ruthenium complexes remains still a reaction of high interest, as reflected by numerous patents and scientific research efforts. Nevertheless, no industrial process has yet been installed, due to the economical (catalyst cost with respect to its productivity) and chemical obstacles mentioned above.

Y. Bouhoute et al. (ACS Catal. 2014, 4, 4232-4241) discloses a supported oxo-tungsten catalyst for the homo-metathesis of isobutylene. Said document does not disclose the claimed ethenolysis process nor the specifically claimed catalysts and in particular the specifically claimed oxo-tungsten catalysts. Document WO 2015/049047 discloses very general oxo-tungsten catalysts but said document does not disclose the specifically claimed catalysts and in particular the specifically claimed oxo-tungsten catalysts. M. P. Conley et al. (Angew. Chem. Int. Ed. 2014, 53, 14221-14224) discloses a supported oxo-tungsten catalyst for the ethenolysis of 2-butenes. Said document does not disclose the specifically claimed catalysts and in particular the specifically claimed oxo-tungsten catalysts.

Ethenolysis of methyl oleate remains a very important reaction to upgrade fatty acids and oils. The products obtained, in particular the alpha-olefins, are widely used as intermediates in many domains (polymerization, perfumery, detergents, lubricants, etc). The cross-metathesis reaction (with ethene) presents supplementary difficulties than the self-metathesis of methyl oleate. The most active system is based on homogeneous ruthenium complexes. However, current performance of this catalytic system is far from industrialization due to the cost of the catalyst with respect to the productivity.

There is thus a need to develop highly active catalysts based on cheaper elements, in particular on elements having an industrial interest. An ultimate system will be based on cheap metals supported on conventional materials allowing recycling and facile separation from the products (starting products and reaction products). From economical and chemical viewpoints, supported molybdenum and tungsten based catalysts are extremely attractive. In the literature, there are relatively few examples describing ethenolysis of methyl oleate on heterogeneous Mo and W catalysts.

### SUMMARY

A first object of the present invention is a process for obtaining alpha-olefins, said process comprising a step of reacting optionally-functionalized internal unsaturated olefins with ethylene in the presence of a supported catalyst selected from a supported oxo-molybdenum or imido-molybdenum catalyst or a supported oxo-tungsten catalyst, preferably selected from a supported oxo-molybdenum catalyst or a supported oxo-tungsten catalyst, said supported oxo-tungsten catalyst being selected from one of the following oxo-tungsten compounds:

□-W(═O)X(CH₂R¹)(CH₂R²) (I)

(□)₂W(═O)(CH₂R¹)(CH₂R²) (III)

said imido-molybdenum catalyst being selected from one of the following imido-molybdenum compounds:

□-Mo(═NR⁴)G(═CHR⁵) (VII)

□-OL^{k}O—Mo(═NR⁴)G(═CHR⁵) (VIII)

wherein,

□ corresponds to a support, “□-” indicates a monopodal catalyst, i.e. a catalyst wherein the metal atom (W or Mo atom) is linked to only one grafting site of the support. “(□)₂” indicates a bipodal catalyst, i.e. a catalyst wherein the metal atom (W or Mo atom) is linked to two grafting sites of the support;

R¹ and R², are independently to each other, selected from hydrogen, linear or branched alkyl groups, —C(CH₃)₃, -Phenyl (Ph), —Si(CH₃)₃, or —C(CH₃)₂Ph, X is selected from aryloxy groups, siloxy groups or pyrolidyl groups,

R⁴ represents a radical selected from aliphatic and aromatic hydrocarbyl radicals, optionally comprising one or more heteroatoms,

R⁵ is selected from hydrogen, linear or branched alkyl groups, —C(CH₃)₃, -Phenyl (Ph), —Si(CH₃)₃, or —C(CH₃)₂Ph,

G is selected from alkoxy groups, aryloxy groups, siloxy groups or pyrolidyl groups,

L^{k} represents a divalent linker.

Preferably, in the catalyst used for the process:
R¹, R² and R⁵, are independently to each other, selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph, and/or
R⁴ represents a radical selected from aliphatic and aromatic hydrocarbyl radicals, optionally comprising one or more heteroatoms, R⁴ comprising from 1 to 36 carbon atoms, preferably from 2 to 28 carbon atoms, more preferably from 3 to 24 carbon atoms,
L^{k} is chosen from a linear, branched or cyclic alkylene, having preferably from 1 to 12 carbon atoms, or an arylene group optionally substituted having preferably from 6 to 12 carbon atoms,
X and G are independently to each other selected from the following groups:

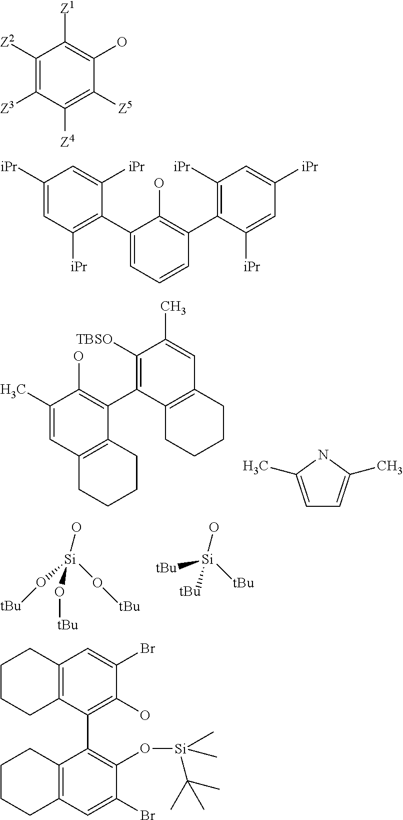

or the radical —O—C(R⁶)₃,

With Z¹, Z², Z³, Z⁴ and Z⁵ are independently to each other selected from hydrogen, methyl, tertio-butyl, adamantyl, mesityl, trifluoromethyl, fluorofluoro, preferably from hydrogen, methyl, tertio-butyl, adamantyl, mesityl, preferably Z²═Z³═Z⁴═H and Z¹ is identical to Z⁵ and is selected from methyl, tertio-butyl, adamantyl, mesityl, and

R⁶ is a linear, branched or cyclic alkyl radical having preferably from 1 to 12 carbon atoms.

According to an embodiment of the invention, the optionally-functionalized internal unsaturated olefins comprise from 8 to 72 carbon atoms, preferably from 8 to 50 carbon atoms, preferably from 10 to 40 carbon atoms, more preferably from 12 to 30 carbon atoms, even more preferably from 14 to 20 carbon atoms. According to an embodiment of the invention, the optionally-functionalized internal unsaturated olefins are functionalized by at least one functional group in terminal position of the mono-olefin. Preferably, the functional group is chosen from ester, acid, amide, amine, alcohol. According to an embodiment of the invention, the optionally-functionalized internal unsaturated olefins are chosen from alkyl oleate.

According to an embodiment of the invention, the support of the catalyst is chosen from silica, modified silica, alumina, modified alumina, titanium oxide, niobium oxide, silica-alumina and organic polymers, such as polystyrene beads. According to an embodiment of the invention, the oxo-molybdenum catalyst does not comprise any carbene function. According to an embodiment of the invention, the oxo-molybdenum catalyst is a monopodal or a bipodal catalyst, preferably a bipodal catalyst.

According to an embodiment of the invention, the supported catalyst is selected from:
the compounds of formula (I): □-W(═O)X(CH₂R¹)(CH₂R²), preferably of formula (Ia):

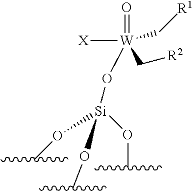

the compounds of formula (II): □-Mo(═O)X(CH₂R¹)(CH₂R²), preferably of formula (IIa):

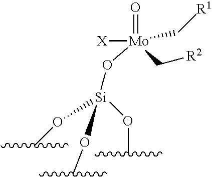

the compounds of formula (III): (□)₂W(═O) (CH₂R¹)(CH₂R²); preferably of formula (IIIa):

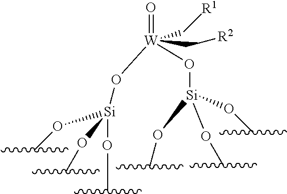

the compounds of formula (IV): (□)₂Mo(═O) (CH₂R¹)(CH₂R²); preferably of formula (IVa):

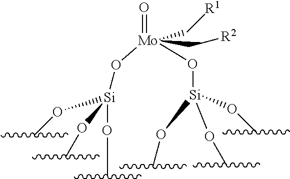

the compounds of formula (VI): (□)₂Mo(═O)(═CHR¹); preferably of formula (Via):

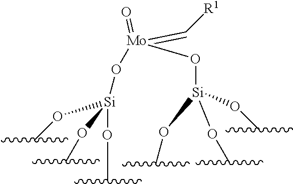

the compounds of formula (VII): □-Mo(═NR⁴)G(═CHR⁵); preferably of formula (VIIa):

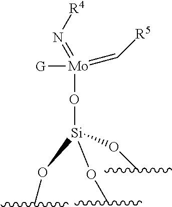

the compounds of formula (VIII): □-OL^{k}O—Mo(═NR⁴)G(═CHR⁵); preferably of formula (Villa):

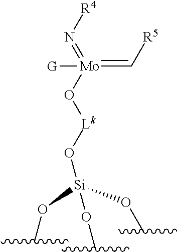

wherein □, X, R¹, R², R⁴, R⁵, G and L^{k} have the same meanings as defined above,

preferably, the supported catalyst is selected from the compounds of formula (I), preferably (Ia), of formula (II), preferably (IIa), of formula (III), preferably (IIIa), of formula (IV), preferably (IVa).

Preferably, the supported catalyst is a compound of formula (III), preferably of formula (IIIa) or a compound of formula (IV), preferably of formula (IVa). According to an embodiment of the invention, the catalyst is obtained by grafting the corresponding complex onto the support □. According to an embodiment of the invention, the reaction is performed at a temperature ranging from 0° C. to 400° C., preferably from 50 to 300° C., more preferably from 100 to 250° C., even more preferably from 120° C. to 200° C.

According to an embodiment of the invention, the reaction is performed at a pressure ranging from 1 to 300 bar, preferably from 3 to 200 bar, more preferably from 5 to 100 bar, even more preferably from 8 to 50 bar. According to an embodiment of the invention, the functionalized internal olefins have a purity of at least 99%. According to an embodiment of the invention, at the beginning of the reaction, the optionally-functionalized internal unsaturated olefins/(W or Mo) molar ratio ranges from 50 to 5000, preferably from 75 to 2000, more preferably from 100 to 1000, even more preferably from 100 to 500. According to an embodiment of the invention, the process further comprises, before the step of reacting, a step of the purification of optionally-functionalized internal unsaturated olefins. According to an embodiment of the invention, the reaction can be performed in the presence of a scavenger, preferably chosen from Al(iBu)₃/SiO₂.

The present invention is also directed to a supported catalyst that can be used in the process of the invention, said supported catalyst being selected from a supported oxo-molybdenum catalyst or a supported oxo-tungsten catalyst or a supported imido-molybdenum catalyst responding to the following formula:

□-W(═O)X(CH₂R¹)(CH₂R²) (I)

□-Mo(═O)X(CH₂R¹)(CH₂R²) (II)

(□)₂W(═O)(CH₂R¹)(CH₂R²) (Ill)

(□)₂Mo(═O)(CH₂R¹)(CH₂R²) (IV)

(□)₂Mo(═O)(═CHR¹) (VI)

□-Mo(═NR⁴)G(═CHR⁵) (VII)

□-OL^{k}O—Mo(═NR⁴)G(═CHR⁵) (VIII)

wherein,

□ corresponds to a support, “□-” indicates a monopodal catalyst, i.e. a catalyst wherein the metal atom (Mo or W atom) is linked to only one grafting site of the support. “(□)₂” indicates a bipodal catalyst, i.e. a catalyst wherein the metal atom (Mo or W atom) is linked to two grafting sites of the support;

R¹ and R², are independently to each other, selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph,

being understood that R¹ and R² cannot be both hydrogen in formula (III);

R⁴ represents a radical selected from aliphatic and aromatic hydrocarbyl radicals, optionally comprising one or more heteroatoms, preferably comprising from 1 to 36 carbon atoms, preferably from 2 to 28 carbon atoms, more preferably from 3 to 24 carbon atoms,

R⁵ is selected from hydrogen, linear or branched alkyl groups, —C(CH₃)₃, -Phenyl (Ph), —Si(CH₃)₃, or —C(CH₃)₂Ph,

G is selected from alkoxy groups, aryloxy groups, siloxy groups or pyrolidyl groups,

L^{k} represents a divalent linker, preferably chosen from a linear, branched or cyclic alkylene, having preferably from 1 to 12 carbon atoms, or an arylene group optionally substituted having preferably from 6 to 12 carbon atoms,

X is selected from aryloxy groups, siloxy groups or pyrolidyl groups,

preferably X and G are independently to each other selected from the following groups:

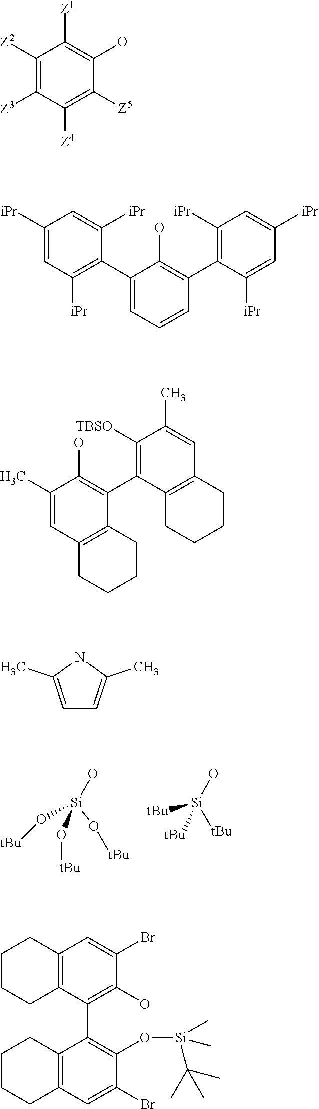

or the radical —O—C(R⁶)₃,

with Z¹, Z², Z³, Z⁴ and Z⁵ are independently to each other selected from hydrogen, methyl, tertio-butyl, adamantyl, mesityl, trifluoromethyl, fluoro, preferably from hydrogen, methyl, tertio-butyl, adamantyl, mesityl, more preferably Z²═Z³═Z⁴═H and Z¹ is identical to Z⁵ and is selected from methyl, tertio-butyl, adamantyl, mesityl,

R⁶ is a linear, branched or cyclic alkyl radical having preferably from 1 to 12 carbon atoms.

The present invention further relates to a method for preparing the supported catalyst of formulas (I), (II), (Ill), (IV), (VI), (VII) and (VIII) according to the invention, said method comprising one of the following reaction schemes:

Reaction scheme 1 for obtaining catalysts of formula (I):

□-OH+W(═O)X(CH₂R¹)(CH₂R²)(CH₂R³)→□-W(═O)X(CH₂R¹)(CH₂R²)

Reaction scheme 1 bis for obtaining catalysts of formula (I):

□-OH+W(═O)(CH₂R¹)(CH₂R²)(CH₂R³)→□W(═O)(CH₂R¹)(CH₂R²)(CH₂R³)

□-W(═O)(CH₂R¹)(CH₂R²)(CH₂R³)+XH→□-W(═O)X(CH₂R¹)(CH₂R²)+R³CH₃

Reaction scheme 2 for obtaining catalysts of formula (II):

□-OH+Mo(═O)X(CH₂R¹)(CH₂R²)(CH₂R³)→□-Mo(═O)X(CH₂R¹)(CH₂R²)

Reaction scheme 2 bis for obtaining catalysts of formula (II):

□-OH+Mo(═O)(CH₂R¹)(CH₂R²)(CH₂R³)→□-Mo(═O)(CH₂R¹)(CH₂R²)(CH₂R³)

□-Mo(═O)(CH₂R¹)(CH₂R²)(CH₂R³)+XH→□-Mo(═O)X(CH₂R¹)(CH₂R²)+R³CH₃

Reaction scheme 3 for obtaining catalysts of formula (III):

(□)₂W(═O)Cl₂+Sn(CH₂R¹)₂(CH₂R²)₂→(□)₂W(═O)(CH₂R¹)(CH₂R²)

Reaction scheme 3 bis for obtaining catalysts of formula (III):

□-OH+□-OH+W(═O)(CH₂R¹)(CH₂R²)(X′)₂→(□)₂W(═O)(CH₂R¹)(CH₂R²)+2X′H

Reaction scheme 4 for obtaining catalysts of formula (IV):

(□)₂Mo(═O)Cl₂+Sn(CH₂R¹)₂(CH₂R²)₂→(□)₂Mo(═O)(CH₂R¹)(CH₂R²)

Reaction scheme 4 bis for obtaining catalysts of formula (IV):

□OH+□-OH+Mo(O)(CH₂R¹)(CH₂R²)(X′)₂→(□)₂Mo(═O)(CH₂R¹)(CH₂R²)+2X′H

Reaction scheme 6 for obtaining catalysts of formula (VI):

□-OH+□-OH+Mo(═O)(═CHR¹)(X″)₂→(□)₂Mo(═O)(═CHR¹)+2X″H

Reaction scheme 7 for obtaining catalysts of formula (VII):

□-OH+Mo(═NR4)(═CHR⁵)(G)₂→(□)Mo(═NR4)G(═CHR⁵)+GH

Reaction scheme 8 for obtaining catalysts of formula (VIII):
□-OL^{k}-OH+Mo(═NR⁴)(═CHR⁵)(G)₂→(□-OL^{k}O)Mo(═NR⁴)G(═CHR⁵)+GH
   wherein

□, X, R¹ R², R⁴, R⁵, G and L^{k} have the same meaning as above regarding the new catalysts of the invention,

R³ is selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R³ is selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph,

X′ and X″ are independently to each other selected from chlorine, bromine, fluorine, aryloxy groups, siloxy groups or pyrolidyl groups, preferably X′ and X″ are independently to each other selected from chlorine, bromine, fluorine or one of the following groups:

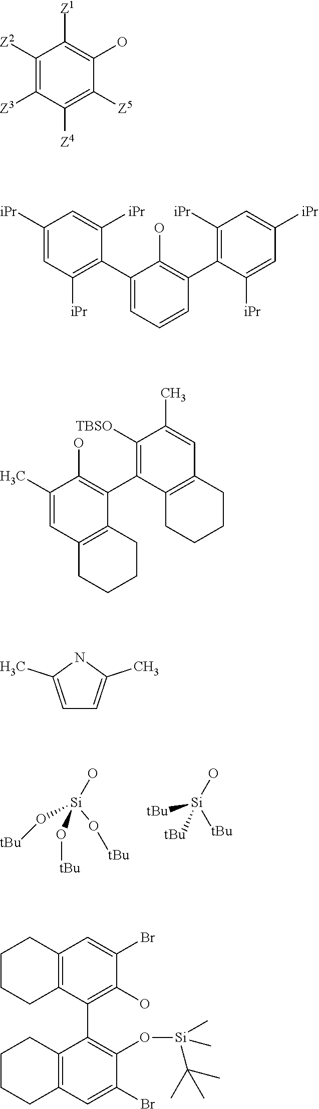

with Z¹, Z², Z³, Z⁴ and Z⁵ are independently to each other selected from hydrogen, methyl, tertio-butyl, adamantyl, mesityl, trifluoromethyl, fluoro, preferably from hydrogen, methyl, tertio-butyl, adamantyl, mesityl, more preferably Z²═Z³═Z⁴═H and Z¹ is identical to Z⁵ and is selected from methyl, tertio-butyl, adamantyl, mesityl.

The present invention also relates to a method for the production of poly-alpha-olefins (PAO), said method comprising:

a) producing alpha-olefins, more particularly C₁₀ alpha-olefins, according to the process of ethenolysis of the invention;

b) oligomerizing the alpha-olefins produced in step a); and

c) optionally hydrogenating the oligomer produced in step b).

According to an embodiment, the poly-alpha-olefins are C₃₀ poly-alpha-olefins, wherein step i) comprises the production of C₁₀ alpha-olefins, preferably 1-decene, and wherein the oligomerization reaction in step ii) is a trimerization reaction. The process of the invention is simple and allows providing desired products with high conversion and a high selectivity, in particular towards the alpha-olefins. Further features and advantages of the invention will appear from the following description of embodiments of the invention, given as non-limiting examples, with reference to the accompanying drawings listed hereunder.

### DETAILED DESCRIPTION

### Process of Ethenolysis Reaction

The present invention is directed to a process for obtaining alpha-olefins, said process comprising a step of reacting internal unsaturated olefins, preferably optionally-functionalized internal mono-unsaturated olefins, more preferably functionalized internal mono-unsaturated olefins, with ethylene in the presence of a supported oxo-molybdenum or imido-molybdenum or oxo-tungsten catalyst,

said oxo-tungsten catalyst being selected from one of the following oxo-tungsten compounds:

□-W(═O)X(CH₂R¹)(CH₂R²) (I)

(□)₂W(═O)(CH₂R¹)(CH₂R²) (III),

said imido-molybdenum catalyst being selected from one of the following imido-molybdenum compounds:

□-Mo(═NR⁴)G(═CHR⁵) (VII)

□-OL^{k}O—Mo(═NR⁴)G(═CHR⁵) (VIII)

wherein,

□ corresponds to a support, “□-” indicates a monopodal catalyst, i.e. a catalyst wherein the metal atom (Mo or W atom) is linked to only one grafting site of the support. “(□)₂” indicates a bipodal catalyst, i.e. a catalyst wherein the metal atom (Mo or W atom) is linked to two grafting sites of the support;

R¹ and R², are independently to each other, selected from hydrogen, linear or branched alkyl groups, the alkyl group preferably having from 1 to 12 carbon atoms, —C(CH₃)₃, -Ph (phenyl), —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph;

R⁴ represents a radical selected from aliphatic and aromatic hydrocarbyl radicals, optionally comprising one or more heteroatoms, preferably comprising from 1 to 36 carbon atoms, preferably from 2 to 28 carbon atoms, more preferably from 3 to 24 carbon atoms, preferably R⁴ is selected from optionally-substituted aryl groups comprising preferably from 6 to 18 carbon atoms, or linear, branched or cyclic alkyl groups, comprising preferably from 1 to 18 carbon atoms, or linear, branched or cyclic alkenyl groups comprising from 2 to 18 carbon atoms,

R⁵ is selected from hydrogen, linear or branched alkyl groups, —C(CH₃)₃, -Phenyl (Ph), —Si(CH₃)₃, or —C(CH₃)₂Ph,

L^{k} represents a divalent linker, for example L^{k} is chosen from an alkylene, linear, branched or cyclic, having for example from 1 to 12 carbon atoms, or an arylene group optionally substituted having for example from 6 to 12 carbon atoms

G is selected from alkoxy groups, aryloxy groups, siloxy groups or pyrolidyl groups,

X is selected from aryloxy groups, siloxy groups or pyrolidyl groups,

preferably X and G are independently to each other selected from the following groups:

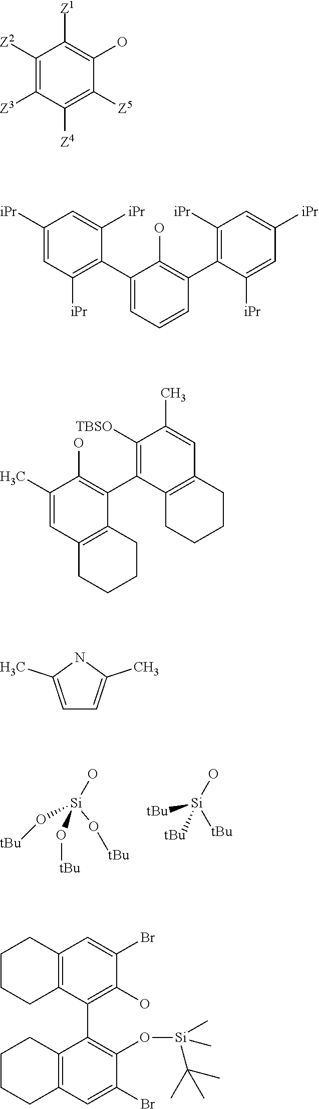

or the radical —O—C(R⁶)₃,

wherein

Z¹, Z², Z³, Z⁴ and Z⁵ are independently to each other selected from hydrogen, methyl, tertio-butyl, adamantyl, mesityl, trifluoromethyl, and fluoro, preferably from hydrogen, methyl, tertio-butyl, adamantyl, mesityl, more preferably Z²═Z³═Z⁴═H and Z¹ is identical to Z⁵ and is selected from methyl, tertio-butyl, adamantyl, mesityl,

R⁶ is a linear, branched or cyclic alkyl radical having preferably from 1 to 12 carbon atoms.

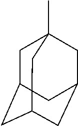

Adamantyl is a (monovalent) group of formula:

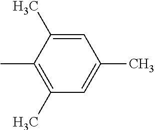

Mesityl is a (monovalent) group of formula:

TBSO is a (monovalent) group of formula:

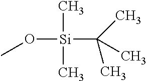

According to an embodiment, X is selected from the following groups:

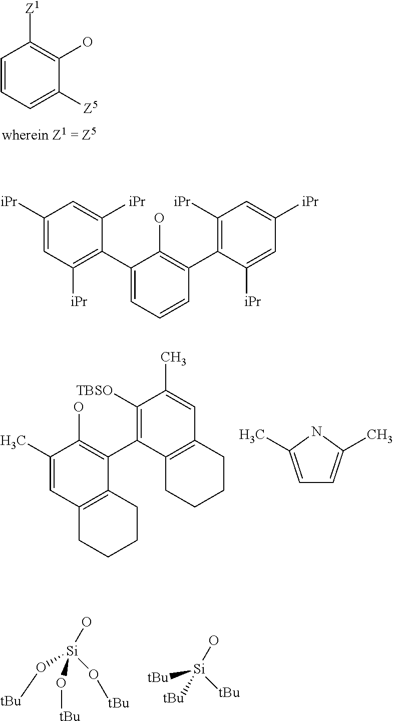

Internal unsaturated olefins, optionally functionalized

The internal unsaturated olefins used in the present invention are olefin compounds comprising at least one carbon-carbon double bond, all the carbon-carbon double bonds being within the hydrocarbon chain of the olefin, i.e. the carbon-carbon double bonds are not in terminal position of the internal unsaturated olefin. The internal unsaturated olefins may be mono-unsaturated or poly-unsaturated. According to a preferred embodiment of the invention, the internal unsaturated olefins are internal mono-unsaturated olefins, i.e. olefins comprising only one carbon-carbon double bond, said carbon-carbon double bond being within the hydrocarbon chain of the olefin, i.e. the carbon-carbon double bond is not in terminal position of the internal mono-unsaturated olefin.

Preferably, the internal unsaturated, in particular mono-unsaturated, olefins are functionalized, preferably in terminal position of the internal mono-unsaturated olefins. The internal unsaturated, in particular mono-unsaturated, olefins may be functionalized by one or more functional groups, preferably by only one functional group. The functional group(s) may be chosen from ester, acid, ether, amide, amine or alcohol.

According to an embodiment of the invention, the optionally-functionalized internal unsaturated, in particular mono-unsaturated, olefins used in the present invention are olefins comprising only one internal carbon-carbon double bond and only one functional group in terminal position of the olefin chain. According to an embodiment of the invention, the internal unsaturated, in particular mono-unsaturated, olefins, optionally functionalized, comprise an unsaturated, in particular a mono-unsaturated, hydrocarbon chain comprising from 8 to 72 carbon atoms, preferably from 8 to 50 carbon atoms, preferably from 10 to 40 carbon atoms, more preferably from 12 to 30 carbon atoms, even more preferably from 14 to 20 carbon atoms. According to an embodiment of the invention, the functionalized internal mono-unsaturated olefins are chosen from alkyl oleates. Preferably, the alkyl group of the alkyl oleate comprises from 1 to 10 carbon atoms, more preferably from 1 to 5 carbon atoms. According to an embodiment of the invention, the internal unsaturated olefins are selected from triglycerides, preferably mono-unsaturated triglycerides. According to an embodiment, the triglycerides, preferably mono-unsaturated triglycerides comprise from 18 to 72 carbon atoms, more preferably from 42 to 66 carbon atoms. The internal poly- or mono-unsaturated olefins, optionally functionalized, may comprise only one kind of internal poly- or mono-unsaturated olefin or a mixture of different internal mono-unsaturated olefins. Preferably, the internal poly- or mono-unsaturated olefins, optionally unsaturated, as starting product of the reaction, comprise only one kind of internal mono- or poly-unsaturated olefin, optionally functionalized. The internal poly- or mono-unsaturated olefins, optionally functionalized, used in the process of the invention may be of natural or synthetic origin. Preferably, the internal poly- or mono-unsaturated olefins, preferably functionalized, are of natural origin, including the olefins produced by microorganisms such as microalgae, bacteria, fungi and yeasts. The internal poly- or mono-unsaturated olefins, optionally functionalized, as starting product may be derived from long-chain natural poly- or monounsaturated fatty acids. Long-chain natural fatty acid is understood to mean an acid resulting from plant or animal sources, including algae, more generally from the plant kingdom, which are thus renewable, comprising at least 10 and preferably at least 14 carbon atoms per molecule.

As examples of such acids, mention may be made of the cis-4-decenoic acid and cis-9-decenoic acid, cis-5-dodecenoic acid, cis-4-dodecenoic acid, cis-9-tetradecenoic acid, cis-5-tetradecenoic acid, cis-4-tetradecenoic acid, cis-9-hexadecenoic acid, cis-9-octadecenoic acid, trans-9-octadecenoic acid, cis-6-octadecenoic acid, cis-11-octadecenoic acid, 12-hydroxy-cis-9-octadecenoic acid, cis-9-eicosenoic acid, cis-11-eicosenoic acid, cis-5-eicosenoic acid, 14-hydroxy-cis-11-eicosenoic acid, cis-11-docosenoic acid and cis-13-docosenoic acid. These various acids may result from the vegetable oils extracted from various plants, such as sunflower, rape, castor oil plant, bladderpod, olive, soya, palm tree, coriander, celery, dill, carrot, fennel or Limnanthes alba or obtained via oleaginous microorganisms. They may also result from the terrestrial or marine animal world and, in the latter case, both in the form of fish or mammals, on the one hand, and of algae, on the other hand.

Oleaginous microorganisms such as microalgae, bacteria, fungi and yeasts are an attractive alternative to higher plants for lipid production, since they can accumulate high levels of lipids without competing with food production and having oil productivity values higher than oilseed crops. Among them, yeasts have emerged as good candidates, because they are easy to cultivate, to manipulate genetically and they have a high lipid accumulation potential. For this reason, improvement of fatty acid (FA) accumulation in yeasts has become a very important topic in recent years and will be probably still of high importance in the next years.

Recently, the economic production of C5 and C6 sugars from waste cellulosic materials has become plausible, making plant sugars derived from lignocellulose a feasible source of renewable feedstocks. Unlike microalgae, yeast cultivation does not require light, which both reduces input costs and enables production 24 h per day. Essential inputs such as phosphorous and nitrogen are also available from waste streams such as waste water, again reducing production costs.

The optionally-functionalized, internal poly- or mono-unsaturated olefins, as starting mixture of reactants in the process of the invention, generally consist essentially of optionally-functionalized internal poly- or mono-unsaturated olefins. Very few impurities may be present in the starting mixture of optionally-functionalized internal poly- or mono-unsaturated olefins. Preferably, the starting mixture of optionally-functionalized internal poly- or mono-unsaturated olefins comprise at least 95% by weight of optionally-functionalized internal poly- or mono-unsaturated olefins, more preferably at least 97% by weight, even more preferably at least 99% by weight, based on the total weight of the starting mixture of optionally-functionalized internal poly- or mono-unsaturated olefins. Therefore, according to an embodiment, before the ethenolysis reaction in the process of the invention, there is a step of purification of the mixture of optionally-functionalized internal poly- or mono-unsaturated olefins, in particular when the optionally-functionalized internal poly- or mono-unsaturated olefins are of natural origin.

Catalyst Used in the Process for the Ethenolysis Reaction

The catalyst used in the present invention in order to perform the ethenolysis reaction is chosen from supported oxo-molybdenum catalysts, oxo-tungsten catalysts, or imido-molybdenum catalysts, and some of them are new products per se as explained hereinafter. Preferably, the catalyst used in the present invention in order to perform the ethonolysis reaction is chosen from supported oxo-molybdenum catalysts or oxo-tungsten catalysts.

By “supported oxo-molybdenum catalyst”, it is to be understood a catalyst comprising a molybdenum atom linked to a support and linked to an oxygen atom with a double bond (oxo). By “supported oxo-tungsten catalyst”, it is to be understood a catalyst comprising a tungsten atom linked to a support and to an oxygen atom with a double bond (oxo). By “supported imido-molybdenum catalyst”, it is to be understood a catalyst comprising a molybdenum atom linked to a support and linked to a nitrogen atom with a double bond (imido).

The supported oxo-tungsten catalyst used in the process for the ethenolysis reaction is selected from:

□-W(═O)X(CH₂R¹)(CH₂R²) (I)

(□)₂W(═O)(CH₂R¹)(CH₂R²) (III)

wherein,

□ corresponds to a support, “□-” indicates a monopodal catalyst, i.e. a catalyst wherein the metal atom (Mo or W atom) is linked to only one grafting site of the support. “(□)₂” indicates a bipodal catalyst, i.e. a catalyst wherein the metal atom (Mo or W atom) is linked to two grafting sites of the support;

R¹ and R², are independently to each other, selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph;

X is selected from aryloxy groups, siloxy groups or pyrolidyl groups, preferably X is selected from the following groups:

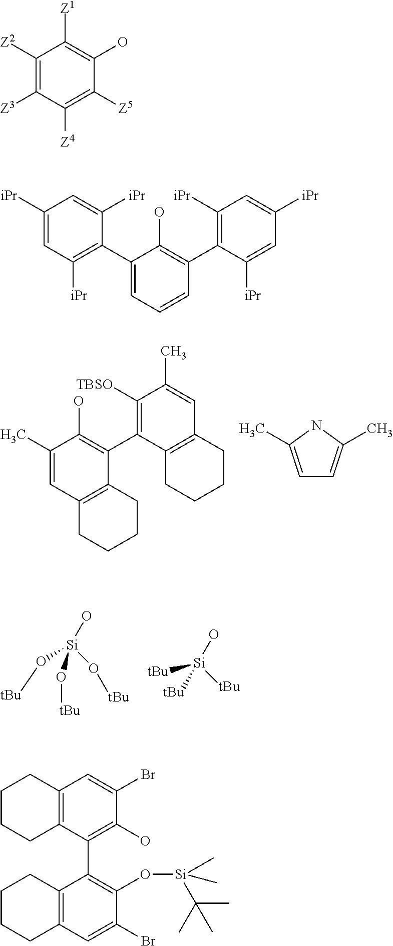

wherein Z¹, Z², Z³, Z⁴ and Z⁵ are as defined above.

As an example, the supported oxo-tungsten catalyst may be selected from one of the following catalysts:

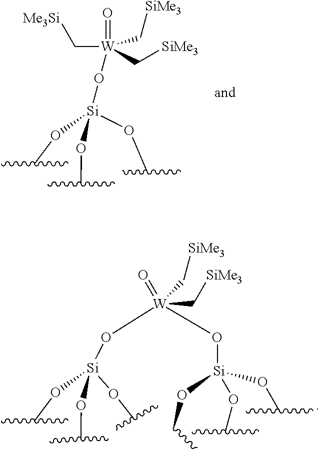

The supported imido-molybdenum catalyst used in the process for ethenolysis of the invention is selected from the catalysts of formula (VII) or (VIII) as defined above. According to an embodiment, in formulas (VII) and (VIII), R⁴ is selected from aryl groups optionally substituted, preferably from aryl groups substituted by at least one, preferably at least two substituents, preferably R⁴ comprises from 6 to 24 carbon atoms, more preferably from 7 to 20 carbon atoms, more preferably from 8 to 16 carbon atoms. According to an embodiment, in formula (VII) and/or in formula (VIII), R⁴ is selected from phenyl, benzyl, 2,6-diisopropylphenyl.

According to a particular embodiment of the invention, the supported catalyst does not comprise carbene. In particular, the molybdenum (Mo) atom, respectively the tungsten (W) atom, is preferably not linked to a carbon atom with a double bond. According an embodiment of the invention, the supported catalyst is a oxo-molybdenum catalyst and the molybdenum atom is linked to ligands selected from methyl, ethyl, propyl, phenyl, tertio-butyl, neosilyl (—CH₂SiMe₃), neophyl (—C₆H₅C(CH₃)₂CH₂), neopentyl (—CH₂C(CH₃)₃). According to an embodiment of the invention, the supported catalyst is a monopodal or a bipodal catalyst, preferably a bipodal catalyst.

By “monopodal catalyst”, it is to be understood a catalyst wherein the metal atom (Mo or W atom) is linked to only one grafting site of the support. By “bipodal catalyst”, it is to be understood a catalyst wherein the metal atom (Mo or W atom) is linked to two grafting sites of the support.

The support is preferably chosen from silica (SiO₂), modified silica, alumina (Al₂O₃), modified alumina, titanium oxide (TiO₂), niobium oxide, silica-alumina and organic polymers, such as polystyrene beads. For example, the silica support may be modified by Lewis acid based on boron, zinc, lanthanide (such as Sc, Y, La), group IV elements (such as Ti, Zr, Hf), group V elements (such as Ta, V, Nb), phenols or hydroquinones. For example, the alumina may be modified by chlorine atoms or by Lewis acid based on boron, zinc, lanthanide (such as Sc, Y, La), group IV elements (such as Ti, Zr, Hf), group V elements (such as Ta, V, Nb). According to an embodiment, the catalyst used for the ethenolysis reaction is of formula (III). Preferably, in formula (III), both R¹ and R² do not represent hydrogen.

According to an embodiment of the invention, the supported catalyst used in the process for the ethenolysis reaction is selected from:
the compounds of formula (I): □-W(═O)X(CH₂R¹)(CH₂R²), preferably of formula (Ia):

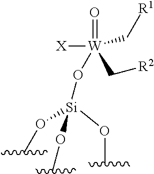

the compounds of formula (II): □-Mo(═O)X(CH₂R¹)(CH₂R²), preferably of formula (IIa):

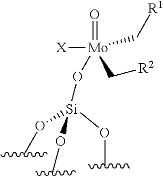

the compounds of formula (III): (□)₂W(═O) (CH₂R¹)(CH₂R²); preferably of formula (IIIa):

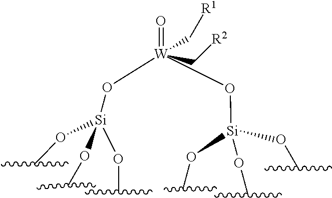

the compounds of formula (IV): (□)₂Mo(═O) (CH₂R¹)(CH₂R²); preferably of formula (IVa):

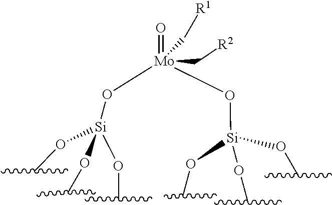

the compounds of formula (VI): (□)₂Mo(═O)(═CHR¹); preferably of formula (Via):

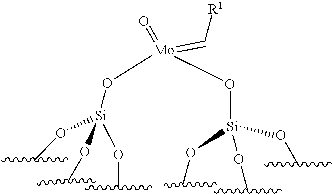

the compounds of formula (VII): □-Mo(═NR⁴)G(═CHR⁵); preferably of formula (VIIa):

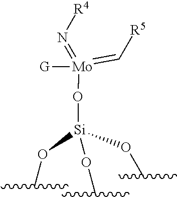

the compounds of formula (VIII): □-OL^{k}O—Mo(═NR⁴)G(═CHR⁵); preferably of formula (Villa):

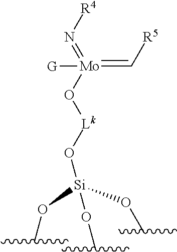

According to a preferred embodiment, the supported catalyst is selected from the compounds of formula (I), preferably (Ia), of formula (II), preferably (IIa), of formula (III), preferably (IIIa), of formula (IV), preferably (IVa).

Catalysts of formula (V) and (Va) are also described in the present application:

(□)₂W(═O)(═CHR¹); formula (V):

formula (Va):

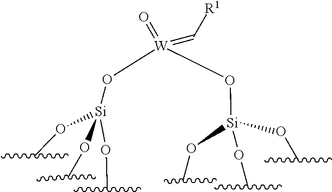

In formulas (I), (Ia), (II), (IIa), (III), (IIIa), (IV), (IVa), (V), (Va), (VI), (Via), (VII), (VIIa), (VIII) and (Villa) defined above:

□ corresponds to a support, “□-” indicates a monopodal catalyst, i.e. a catalyst wherein the metal atom (Mo or W atom) is linked to only one grafting site of the support. “(□)₂” indicates a bipodal catalyst, i.e. a catalyst wherein the metal atom (Mo or W atom) is linked to two grafting sites of the support;

R¹ and R², are independently to each other, selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph;

R⁴ is a radical selected from aliphatic and aromatic hydrocarbyl radicals, optionally comprising one or more heteroatoms, preferably comprising from 1 to 36 carbon atoms, preferably from 2 to 28 carbon atoms, more preferably from 3 to 24 carbon atoms, preferably R⁴ is selected from optionally-substituted aryl groups comprising preferably from 6 to 18 carbon atoms, or linear, branched or cyclic alkyl groups, comprising preferably from 1 to 18 carbon atoms, or linear, branched or cyclic alkenyl groups comprising from 2 to 18 carbon atoms;

R⁵ is selected from hydrogen, linear or branched alkyl groups, —C(CH₃)₃, -Phenyl (Ph), —Si(CH₃)₃, or —C(CH₃)₂Ph, preferably from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph;

L^{k} represents a divalent linker, for example L^{k} is chosen from an alkylene, linear, branched or cyclic, having for example from 1 to 12 carbon atoms, or an arylene group optionally substituted having for example from 6 to 12 carbon atoms; X is selected from aryloxy groups, siloxy groups or pyrolidyl groups, preferably X is selected from the following groups:

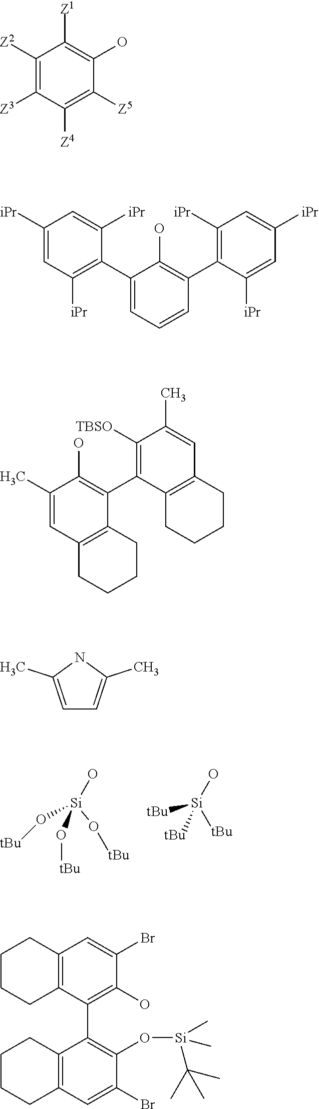

with Z is selected from methyl, tertio-butyl, adamantyl, mesityl, trifluoromethyl, fluoro, preferably Z²═Z³═Z⁴═H and Z¹ is identical to Z⁵ and is selected from methyl, tertio-butyl, adamantyl, mesityl,

G is selected from alkoxy groups, aryloxy groups, siloxy groups or pyrolidyl groups, preferably G is one of the groups defined for X.

According to an embodiment, in formulas (VII), (VIIa), (VIII) and (Villa), R⁴ is selected from aryl groups optionally substituted, preferably from aryl groups substituted by at least one, preferably at least two substituents, preferably R⁴ comprises from 6 to 24 carbon atoms, more preferably from 7 to 20 carbon atoms, more preferably from 8 to 16 carbon atoms. According to an embodiment, in formula (VII) and/or in formula (VIII), R⁴ is selected from phenyl, benzyl, 2,6-diisopropylphenyl. According to an embodiment, the catalyst used for the ethenolysis reaction is of formula (IIIa). Preferably, in formula (IIIa), both R¹ and R² do not represent hydrogen.

The supported catalyst may be obtained by a method such as described in the “method for preparing the catalysts” part below and in the examples. In particular, the method for preparing the catalyst of the invention comprises one of the following reaction schemes:

Reaction scheme 1 for obtaining catalysts of formula (I):

□-OH+W(═O)X(CH₂R¹)(CH₂R²)(CH₂R³)→□-W(═O)X(CH₂R¹)(CH₂R²)

Reaction scheme 1bis for obtaining catalysts of formula (I):

□-OH+W(═O)(CH₂R¹)(CH₂R²)(CH₂R³)→□-W(═O)(CH₂R¹)(CH₂R²)(CH₂R³)

□-W(═O)(CH₂R¹)(CH₂R²)(CH₂R³)+XH→□-W(═O)X(CH₂R¹)(CH₂R²)+R³CH₃

Reaction scheme 2 for obtaining catalysts of formula (II):

□-OH+Mo(═O)X(CH₂R¹)(CH₂R²)(CH₂R³)→□—Mo(═O)X(CH₂R¹)(CH₂R²)

Reaction scheme 2bis for obtaining catalysts of formula (II):

□-OH+Mo(═O)(CH₂R¹)(CH₂R²)(CH₂R³)→□-Mo(═O)(CH₂R¹)(CH₂R²)(CH₂R³)

□-Mo(═O)(CH₂R¹)(CH₂R²)(CH₂R³)+XH→□-Mo(═O)X(CH₂R¹)(CH₂R²)+R³CH₃

Reaction scheme 3 for obtaining catalysts of formula (III):

□-OH+□-OH+W(═O)(CH₂R¹)(CH₂R²)(X′)₂→(□)₂W(═O)(CH₂R¹)(CH₂R²)+2X′H

Reaction scheme 3bis for obtaining catalysts of formula (III):

(□)₂W(═O)Cl₂+Sn(CH₂R¹)₂(CH₂R²)→(□)₂W(═O)(CH₂R¹)(CH₂R²)

Reaction scheme 4 for obtaining catalysts of formula (IV):

□-OH+□-OH+Mo(═O)(CH₂R¹)(CH₂R²)(X′)₂→(O)₂Mo(═O)(CH₂R¹)(CH₂R²)+2X′H

Reaction scheme 4bis for obtaining catalysts of formula (IV):

(□)₂MO(═O)Cl₂+Sn(CH₂R¹)₂(CH₂R²)₂→(□)₂Mo(═O)(CH₂R¹)(CH₂R²)

Reaction scheme 6 for obtaining catalysts of formula (VI):

□-OH+□-OH+Mo(═O)(═CHR¹)(X″)₂→(□)₂Mo(═O)(═CHR¹)+2X″H

Reaction scheme 7 for obtaining catalysts of formula (VII):

□-OH+Mo(═NR4)(═CHR⁵)(G)₂→(□)Mo(═NR4)G(═CHR⁵)+GH

Reaction scheme 8 for obtaining catalysts of formula (VIII):

□-OL^{k}-OH+Mo(═NR⁴)(═CHR⁵)(G)₂→(□-OL^{k}O)Mo(═NR⁴)G(═CHR⁵)+GH

wherein

□, X, R¹ and R², L^{k}, R⁴, R⁵ and G have the same meaning as in formulas (I), (II), (III), (IV), (V), (VI), (VII) and (VIII),

R³ is selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R³ is selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph,

X′ and X″ are independently to each other selected from chlorine, bromine, fluorine, aryloxy groups, siloxy groups or pyrolidyl groups, preferably X′ and X″ are selected from chlorine, bromine, fluorine or one of the following groups:

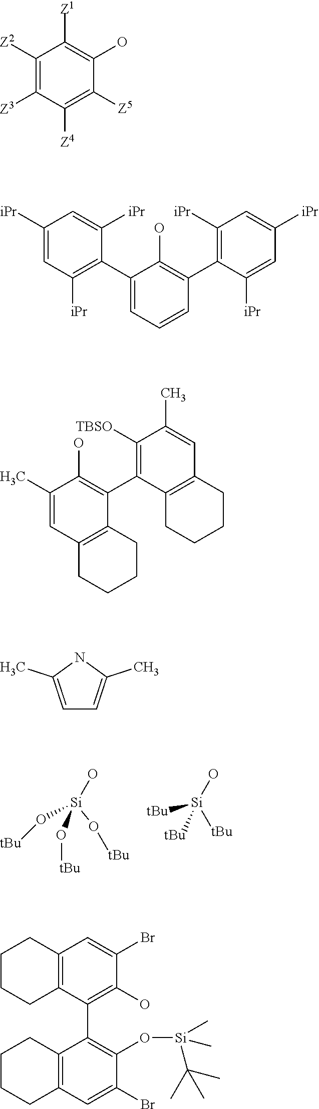

with Z¹, Z², Z³, Z⁴ and Z⁵ are independently to each other selected from hydrogen, methyl, tertio-butyl, adamantyl, mesityl, trifluoromethyl, fluoro, preferably Z²═Z³═Z⁴═H and Z¹ is identical to Z⁵ and is selected from methyl, tertio-butyl, adamantyl, mesityl.

According to an embodiment, the catalyst used in the process of the invention is obtained by grafting the corresponding complex onto the support □. For example, the catalyst used in the process of the invention may be obtained according to one of the following reaction schemes:

Reaction scheme 1 for obtaining catalysts of formula (I):

□-OH+W(═O)X(CH₂R¹)(CH₂R²)(CH₂R³)→□-W(═O)X(CH₂R¹)(CH₂R²)

Reaction scheme 2 for obtaining catalysts of formula (II):

□-OH+Mo(═O)X(CH₂R¹)(CH₂R²)(CH₂R³)→□-Mo(═O)X(CH₂R¹)(CH₂R²)

Reaction scheme 3 for obtaining catalysts of formula (III):

□-OH+□-OH+W(═O)(CH₂R¹)(CH₂R²)(X′)₂→(□)₂W(═O)(CH₂R¹)(CH₂R²)+2X′H

Reaction scheme 4 for obtaining catalysts of formula (IV):

(□)₂Mo(═O)Cl₂+Sn(CH₂R¹)₂(CH₂R²)₂→(□)₂Mo(═O)(CH₂R¹)(CH₂R²)

Reaction scheme 4 for obtaining catalysts of formula (IV):

□-OH+□-OH+Mo(═O)(CH₂R¹)(CH₂R²)(X′)₂→(□)₂Mo(═O)(CH₂R¹)(CH₂R²)+2X′H

Reaction scheme 6 for obtaining catalysts of formula (VI):

□-OH+□-OH+Mo(═O)(═CHR¹)(X″)₂→(□)₂Mo(═O)(═CHR′)+2X″H

Reaction scheme 7 for obtaining catalysts of formula (VII):

□-OH+Mo(═NR4)(═CHR⁵)(G)₂→(□)Mo(═NR4)G(═CHR⁵)+GH

Reaction scheme 8 for obtaining catalysts of formula (VIII):

□-OL^{k}-OH+Mo(═NR⁴)(═CHR⁵)(G)₂→(□-OL^{k}O)Mo(═NR⁴)G(═CHR⁵)+GH

According to an embodiment of the invention, the catalyst used in the process of the invention is a catalyst of formula (IIIa), in particular a catalyst of formula (IIIa) obtained by the following reaction scheme:

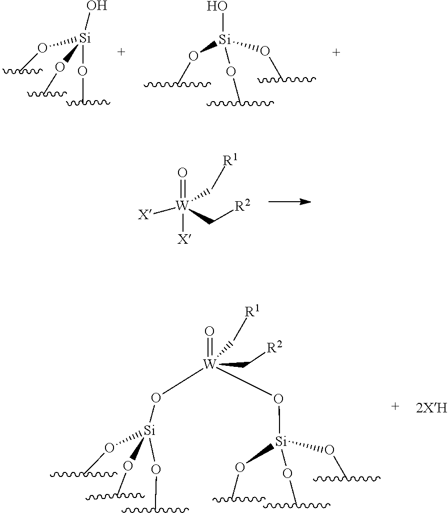

wherein X′ is chosen from chlorine, bromine, fluorine, aryloxy groups, siloxy groups or pyrolidyl groups, preferably X′ is selected from chlorine, bromine, fluorine or one of the following groups:

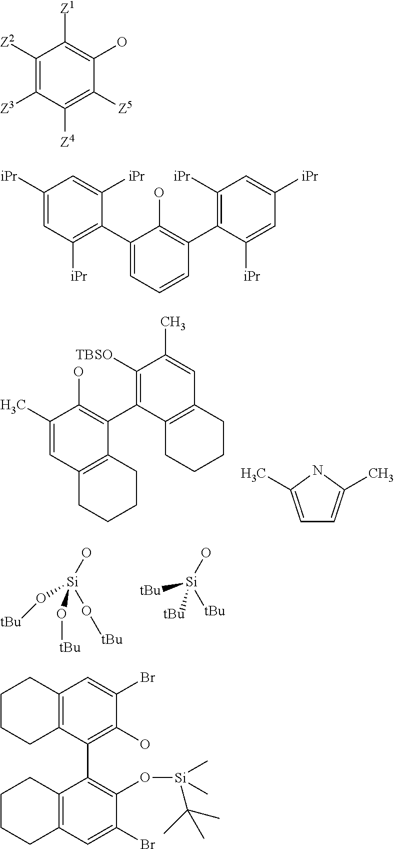

with Z¹, Z², Z³, Z⁴ and Z⁵ are as defined above.

According to an embodiment of the invention, the catalyst is activated before the ethenolysis reaction. Preferably, the activation is performed by addition of an alkylating agent. As an example of alkylating agent, mention may be made of SnBu₄, SnMe₄. The alkylating agent may be introduced in excess during the catalyst preparation and/or at the beginning of the ethenolysis reaction. Preferably, the molar ratio Sn/(W or Mo) may range from 1 to 100.

Ethenolysis Reaction

The process of the present invention comprises a step of reaction between optionally-functionalized internal unsaturated, in particular mono-unsaturated, olefins and ethylene in the presence of a supported oxo-Mo or imido-Mo or oxo-W based catalyst in order to produce alpha-olefins. Said reaction is a metathesis reaction known as ethenolysis reaction. Preferably, the ethenolysis reaction is performed in the presence of a supported oxo-Mo or oxo-W based catalyst.

The process of the present invention leads to reaction products comprising alpha-olefins and optionally functionalized alpha-olefins. Indeed, if the internal mono-unsaturated olefin used as a reactant of the ethenolysis reaction is functionalized, the reaction products comprise alpha-olefins and functionalized alpha-olefins. In order to isolate the alpha-olefins (which is not functionalized), there may be a step of separation of the reaction products, for example by distillation.

When the reaction products are designed:

By “alpha-olefins”, it is to be understood an olefin consisting in carbon and hydrogen atoms and comprising one carbon-carbon double bond in terminal position of the olefin chain and optionally at least one other carbon-carbon double bond. In particular, when the starting olefin is mono-unsaturated, the product “alpha-olefin” comprises only one carbon-carbon double bond in terminal position.

By “functionalized alpha-olefins”, it is to be understood an olefin comprising at least one carbon-carbon double atom in terminal position of the olefin chain and one functional group at the other terminal position. In particular, when the starting functionalized olefin is mono-unsaturated, the product “functionalized alpha-olefin” comprises only one carbon-carbon double bond in terminal position of the olefin and one functional group at the other terminal position.

According to an embodiment of the invention, the reaction is performed at a temperature ranging from 0° C. to 400° C., preferably from 50 to 300° C., more preferably from 100 to 250° C., even more preferably from 120° C. to 200° C. According to an embodiment of the invention, when the catalyst is selected from imido-molybdenum catalysts, then the reaction is preferably performed at a temperature less than or equal to 200° C., more preferably less than or equal to 100° C., even more preferably less than or equal to 75° C. Indeed, a lower temperature allows decreasing the risk of isomerization of the products of the ethenolysis reaction.

According to an embodiment of the invention, the reaction is performed at a pressure ranging from 0.5 to 300 bar, preferably from 1 to 300 bar, preferably from 3 to 200 bar, more preferably from 5 to 100 bar, even more preferably from 8 to 50 bar. According to an embodiment of the invention, the optionally-functionalized internal mono-unsaturated olefins/(Mo or W) molar ratio at the beginning of the reaction ranges from 50 to 5000, preferably from 75 to 2000, more preferably from 100 to 1000, even more preferably from 100 to 500.

According to an embodiment of the invention, the step of reacting is performed in the presence of a solvent. Among solvents that can be used during the ethenolysis reaction, mention may be made of toluene, heptane or xylenes.

According to an embodiment of the invention, the step of reacting is performed in the presence of a scavenger. Indeed, the scavenger allows removing impurities. The scavenger may be chosen from Al(iBu)₃/SiO₂. “iBu” refers to iso-butyl. Preferably, the molar ratio between the amount of optionally-functionalized mono-unsaturated olefin and the amount of the aluminum on surface may ranges from 1 to 10000.

The process of the invention provides high rate of conversion. The rate of conversion in percentage is defined as follows:

100×(% mol of optionally-functionalized unsaturated olefin at the beginning of the reaction−% mol of optionally-functionalized unsaturated olefin at the end of the reaction process)/(% mol of optionally-functionalized unsaturated olefin at the beginning of the reaction).

The process of the invention is very selective, i.e. the process of the invention leads in majority to the products of the cross-metathesis reaction. Otherwise, for example a homo-metathesis reaction could occur if the optionally-functionalized mono-unsaturated olefin reacts with itself. The process of the invention with the specific catalyst allows providing in majority (i.e. in a quantity of more than 50% by mole based on the total amount by mole of reaction products) the products of the cross-metathesis reaction including alpha-olefins.

The molar selectivity of ethenolysis in percentage may be calculated as follows:

100×[“mol of alpha-olefins”+“mol of functionalized alpha-olefins”]/“mol of reaction products”.

The “mol of alpha-olefins” is the amount of alpha-olefins at the end of the reaction expressed in mol.

The “mol of functionalized alpha-olefins” is the amount of functionalized alpha-olefins at the end of the reaction expressed in mol. The “mol of reaction products” is the total amount of the products obtained at the end of the reaction expressed in mol. The reaction products may comprise the liquid products present in the reaction medium, in particular the alpha-olefins obtained at the end of the reaction, the functionalized alpha-olefins obtained at the end of the reaction, but also product(s) obtained from the homo-metathesis of the optionally-functionalized unsaturated olefin.

Preferably, the selectivity of the process of the invention is equal to or higher than 70%, preferably equal to or higher than 75%, more preferably equal to or higher than 80%, even more preferably equal to or higher than 85%, still more preferably equal to or higher than 90%, ideally equal to or higher than 95%. Preferably, ethylene is introduced in stoichiometric excess during the ethenolysis reaction, as compared with the optionally-functionalized unsaturated olefin.

The produced alpha-olefins, more particularly the C₁₀ alpha-olefins produced according to the process of the invention (such as 1-decene), can be used as or converted into a fuel, in particular a biofuel. These alpha-olefins, more particularly C₁₀ alpha-olefins produced according to the invention (such as 1-decene), can also be used as starting material for the production of chemicals or personal care additives (e.g. polymers, surfactants, plastics, textiles, solvents, adhesives, etc.). They can also be used as feedstock for subsequent reactions, such as hydrogenation and/or oligomerization reactions, to make other products.

### Method for the Production of Poly-Alpha-Olefins (PAOs)

A further aspect of the invention relates to a method for the production of poly-alpha-olefins (PAO), said method comprising:

a) producing alpha-olefins, more particularly C₁₀ alpha-olefins, according to the process of ethenolysis according to the present invention;

b) oligomerizing the alpha-olefins produced in step a); and

c) optionally hydrogenating the oligomer produced in step b).

According to an embodiment, the method for the production of poly-alpha-olefins (PAO) leads to the production of C30 PAOs, and comprises:

a) producing C₁₀ alpha-olefins, preferably 1-decene, according to the process of ethenolysis according to the invention;

b) trimerizing the C10 alpha-olefins produced in step a); and

c) optionally hydrogenating the trimer produced in step b).

Oligomerization of alpha-olefins in the presence of a catalyst, in particular a C₁₀ alpha-olefin such as 1-decene, is well known in the art. Catalysts that can be used for the oligomerization step are for example, but not limited to, AlCl₃, BF₃, BF₃ complexes for cationic oligomerization, and metal based catalysts like metallocenes. Following the oligomerization step, residual unsaturation that is potentially present in the oligomers can be saturated by catalytic hydrogenation resulting in saturated aliphatic hydrocarbons with one or more side branches.

The oligomers obtained by methods as described herein are known under the generic name of poly-alpha-olefins (PAO). The PAOs, more particularly the C₃₀ PAOs, obtainable by a method as described herein can be used as base oils, which display very attractive viscosity indices, with the viscosity increasing with the number of carbons. These base oils can be used, together with additives and optionally other base oils, to formulate lubricants. In particular, PAOs with a number of carbons of about 30 to 35, in particular 30, are preferred for automotive lubricants.

### Catalyst

The present invention also concerns new catalysts that can be used in the process of the invention. The new catalyst of the invention is selected from the following compounds:

□-W(═O)X(CH₂R¹)(CH₂R²) (I)

□-Mo(═O)(CH₂R¹)(CH₂R²) (II)

(□)₂W(═O)(CH₂R¹)(CH₂R²) (III)

(□)₂Mo(═O)(CH₂R¹)(CH₂R²) (IV)

(□)₂Mo(═O)(═CHR′) (VI)

□-Mo(═NR⁴)G(═CHR⁵) (VII)

□-OL^{k}O—Mo(═NR⁴)G(═CHR⁵) (VIII)

wherein,

□ corresponds to a support, “□-” indicates a monopodal catalyst, i.e. a catalyst wherein the metal atom (Mo or W atom) is linked to only one grafting site of the support. “(□)₂” indicates a bipodal catalyst, i.e. a catalyst wherein the metal atom (Mo or W atom) is linked to two grafting sites of the support;

R¹ R² and R⁵, are independently to each other, selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph, being understood that R¹ and R² cannot be both hydrogen in formula (III);

R⁴ represents a radical selected from aliphatic and aromatic hydrocarbyl radicals, optionally comprising one or more heteroatoms, preferably comprising from 1 to 36 carbon atoms, preferably from 2 to 28 carbon atoms, more preferably from 3 to 24 carbon atoms, preferably R⁴ is selected from optionally-substituted aryl groups comprising preferably from 6 to 18 carbon atoms, or linear, branched or cyclic alkyl groups, comprising preferably from 1 to 18 carbon atoms, or linear, branched or cyclic alkenyl groups comprising from 2 to 18 carbon atoms,

L^{k} represents a divalent linker, for example L^{k} is chosen from an alkylene, linear, branched or cyclic, having for example from 1 to 12 carbon atoms, or an arylene group optionally substituted having for example from 6 to 12 carbon atoms, G is selected from alkoxy groups, aryloxy groups, siloxy groups or pyrolidyl groups,

X is selected from aryloxy groups, siloxy groups or pyrolidyl groups,

preferably X and G are selected from the following groups:

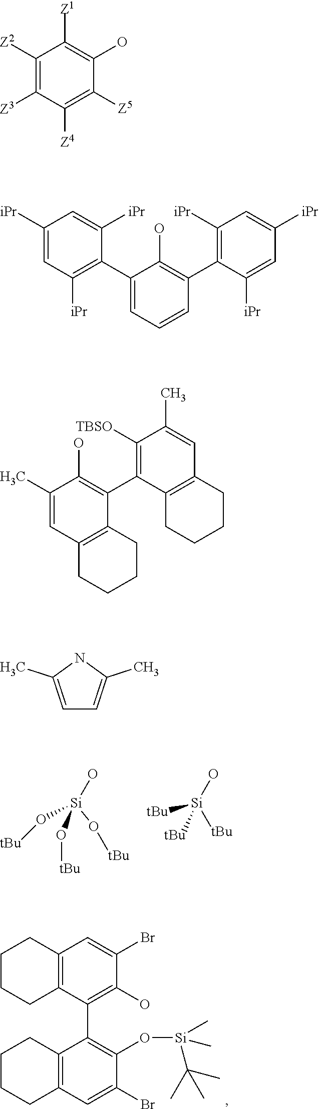

or the radical —O—C(R⁶)₃, with R⁶ is a linear, branched or cyclic alkyl radical having preferably from 1 to 12 carbon atoms,

and preferably from:

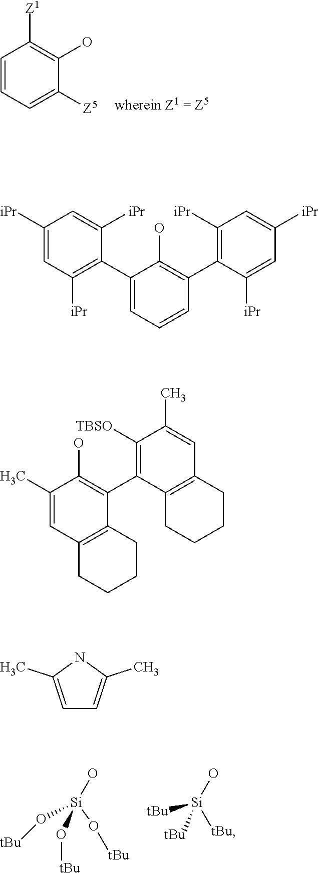

wherein Z¹, Z², Z³, Z⁴ and Z⁵ are independently to each other selected from hydrogen, methyl, tertio-butyl, adamantyl, mesityl, trifluoromethyl, fluoro, preferably from hydrogen, methyl, tertio-butyl, adamantyl, mesityl, more preferably Z²═Z³═Z⁴═H and Z¹ is identical to Z⁵ and is selected from methyl, tertio-butyl, adamantyl, mesityl.

According to an embodiment, in formulas (VII) and (VIII), R⁴ is selected from aryl groups optionally substituted, preferably from aryl groups substituted by at least one, preferably at least two substituents, preferably R⁴ comprises from 6 to 24 carbon atoms, more preferably from 7 to 20 carbon atoms, more preferably from 8 to 16 carbon atoms. According to an embodiment, in formula (VII) and/or in formula (VIII), R⁴ is selected from phenyl, benzyl, 2,6-diisopropylphenyl.

The support □ is preferably chosen from silica (SiO₂), modified silica, alumina (Al₂O₃), modified alumina, titanium oxide (TiO₂), niobium oxide, silica-alumina and organic polymers, such as polystyrene beads. For example, the silica support may be modified by Lewis acid based on boron, zinc, lanthanide (such as Sc, Y, La), group IV elements (such as Ti, Zr, Hf), group V elements (such as Ta, V, Nb), phenols or hydroquinones. For example, the alumina may be modified by chlorine atoms or by Lewis acid based on boron, zinc, lanthanide (such as Sc, Y, La), group IV elements (such as Ti, Zr, Hf), group V elements (such as Ta, V, Nb).

According to a preferred embodiment, the support □ is a silica or a modified silica support. Preferably, the catalyst of the invention comprises and/or consists in one of the following compounds:

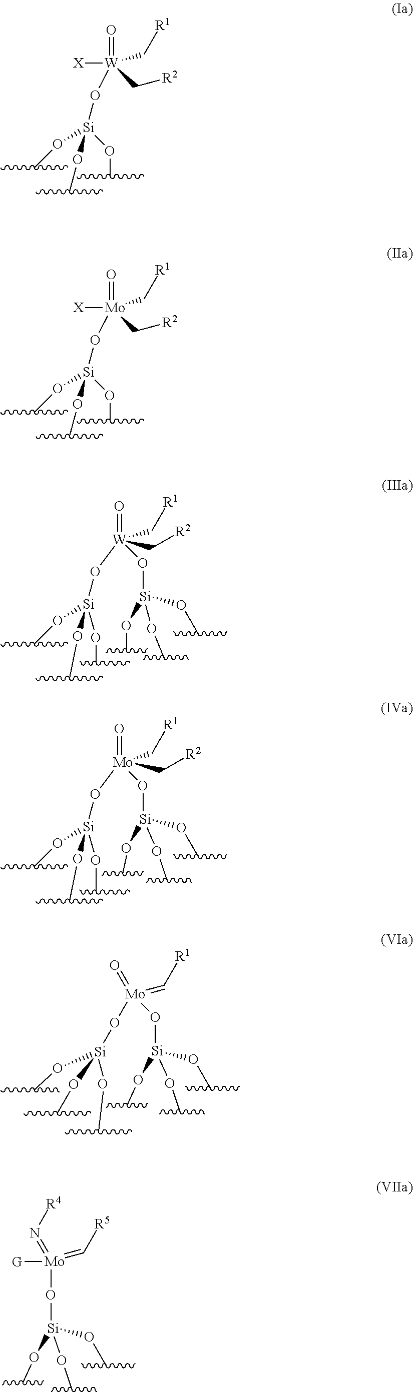

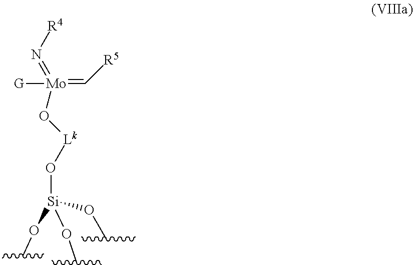

In formulas (Ia), (IIa), (IIIa), (IVa), (VIa) and (VIIa), X, R¹, R², R⁴ and L^{k} have the same meanings as in formulas (I), (II), (III), (IV), (VI), (VII) and (VIII), being understood that R¹ and R² cannot be both hydrogen in formula (IIIa). According to a particular embodiment of the invention, the process for obtaining alpha-olefin according to the invention is performed with the new catalysts according to the invention.

### Method for Preparing the Catalysts

The present invention is also directed to a method for the preparation of the new catalysts of formulas (I), (II), (Ill), (IV)(VI), (VII) and (VIII) according to the invention, said method comprising one of the following reactions:

Reaction scheme 1 for obtaining catalysts of formula (I):

□-OH+W(═O)X(CH₂R¹)(CH₂R²)(CH₂R³)→□-W(═O)X(CH₂R¹)(CH₂R²)

Reaction scheme 1bis for obtaining catalysts of formula (I):

□-OH+W(═O)(CH₂R¹)(CH₂R²)(CH₂R³)→-□W(═O)(CH₂R¹)(CH₂R²)(CH₂R³)

□-W(═O)(CH₂R¹)(CH₂R²)(CH₂R³)+XH→□-W(═O)X(CH₂R¹)(CH₂R²)+R³CH₃

Reaction scheme 2 for obtaining catalysts of formula (II):

□-OH+Mo(═O)X(CH₂R¹)(CH₂R²)(CH₂R³)→□-Mo(═O)X(CH₂R¹)(CH₂R²)

Reaction scheme 2bis for obtaining catalysts of formula (II):

□-OH+Mo(═O)(CH₂R¹)(CH₂R²)(CH₂R³)→□-Mo(═O)(CH₂R¹)(CH₂R²)(CH₂R³)

□-Mo(═O)(CH₂R¹)(CH₂R²)(CH₂R³)+XH→□-Mo(═O)X(CH₂R¹)(CH₂R²)+R³CH₃

Reaction scheme 3 for obtaining catalysts of formula (III):

□-OH+□-OH+W(═O)(CH₂R¹)(CH₂R²)(X′)₂→(□)₂W(═O)(CH₂R¹)(CH₂R²)+2X′H

Reaction scheme 3bis for obtaining catalysts of formula (III):

(□)₂W(═O)Cl₂+Sn(CH₂R¹)₂(CH₂R²)₂→(□)₂W(═O)(CH₂R¹)(CH₂R²)

Reaction scheme 4 for obtaining catalysts of formula (IV):

□-OH+□-OH+Mo(═O)(CH₂R¹)(CH₂R²)(X′)₂→(□)₂Mo(═O)(CH₂R¹)(CH₂R²)+2X′H

Reaction scheme 4bis for obtaining catalysts of formula (IV):

(□)₂Mo(═O)Cl₂+Sn(CH₂R¹)₂(CH₂R²)₂→(□)₂Mo(═O)(CH₂R¹)(CH₂R²)

Reaction scheme 6 for obtaining catalysts of formula (VI):

□-OH+□-OH+Mo(═O)(═CHR¹)(X″)₂→(□)₂Mo(═O)(═CHR′)+2X″H

Reaction scheme 7 for obtaining catalysts of formula (VII):

□-OH+Mo(═NR4)(═CHR⁵)(G)₂→(□)Mo(═NR4)G(═CHR⁵)+GH

Reaction scheme 8 for obtaining catalysts of formula (VIII):

□-OL^{k}-OH+Mo(═NR⁴)(═CHR⁵)(G)₂→(□-OL^{k}O)Mo(═NR⁴)G(═CHR⁵)+GH

In the above reaction schemes,

□, X, R¹, R², R⁴, R⁵, G and L^{k} have the same meaning as in formulas (I), (II), (Ill), (IV), (VI), (VII) and (VIII),

R³ is selected from hydrogen, linear or branched alkyl groups, the alkyl group preferably having from 1 to 12 carbon atoms, —C(CH₃)₃, -Ph, —Si(CH₃)₃, —C(CH₃)₂Ph, preferably R³ is selected from —H, methyl, ethyl, propyl, isopropyl, butyl, isobutyl, pentyl, isopentyl, n-hexyl, —C(CH₃)₃, -Ph, —Si(CH₃)₃, —C(CH₃)₂Ph,

X′ and X″ are independently to each other selected from chlorine, bromine, fluorine, aryloxy groups, siloxy groups or pyrolidyl groups, preferably X′ and X″ are selected from chlorine, bromine, fluorine or one of the following groups:

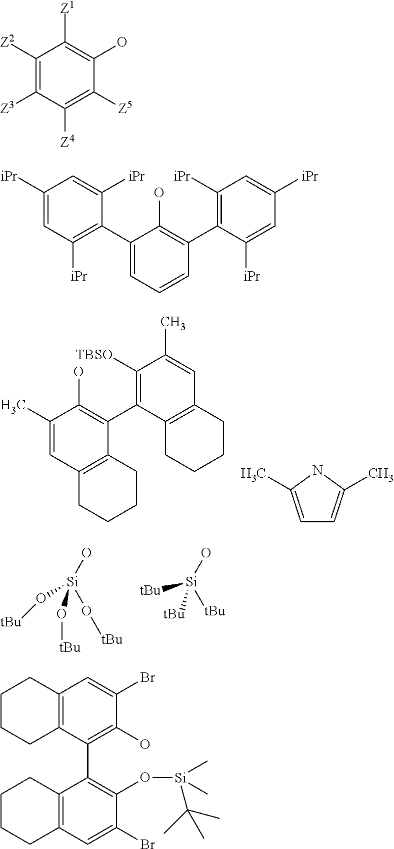

with Z¹, Z², Z³, Z⁴ and Z⁵ are independently to each other selected from hydrogen, methyl, tertio-butyl, adamantyl, mesityl, trifluoromethyl, fluoro, more preferably Z²═Z³═Z⁴═H and Z¹ is identical to Z⁵ and is selected from methyl, tertio-butyl, adamantyl, mesityl.

The catalysts of the invention may be prepared according to one of the above-defined reaction scheme in a solvent, such as pentane, hexane, heptane, toluene, chlorobenzene or ether. The catalysts of the invention may be prepared at a temperature ranging from 20° C. to 80° C., preferably from 20° C. to 50° C., around 25° C. The catalysts of the invention may be prepared at a pressure of about 1 bar of argon or nitrogen (N₂).

According to an embodiment, the molar ratio between the amount of tungsten or molybdenum and the amount of the OH group linked to the support ranges from 1 to 100. Preferably, the molar ratio between the tungsten and the OH group linked to the support ranges from 1 to 2 for the reaction schemes 1, 1 bis, 2, 2bis, 3, 4, 5 and 6.

The compound of formula (Ia) may be prepared in pentane, hexane, heptane, toluene or chlorobenzene solvent at a temperature ranging from 20° C. to 80° C. according to one of the following reaction schemes:

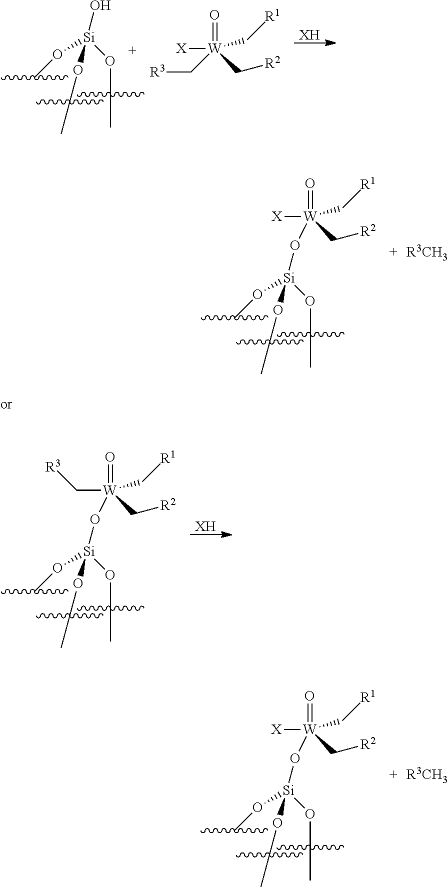

wherein R¹, R², R³ and X have the same meaning as defined for the catalyst of formula (I).

The silica support may for example be dehydroxylated at a high temperature (around 700° C.) before grafting the corresponding complex onto the silica support. The high temperature for the dehydroxylation facilitates the formation of a monopodal catalyst. Compounds of formula (IIa) may be prepared according to a similar method as the method for preparing compounds of formula (Ia), by replacing the tungsten atom by a molybdenum atom.

Compounds of formula (IIIa) may be prepared in pentane, hexane, heptane, toluene or chlorobenzene solvent at a temperature ranging from 20° C. to 80° C. according to one of the following reaction schemes:

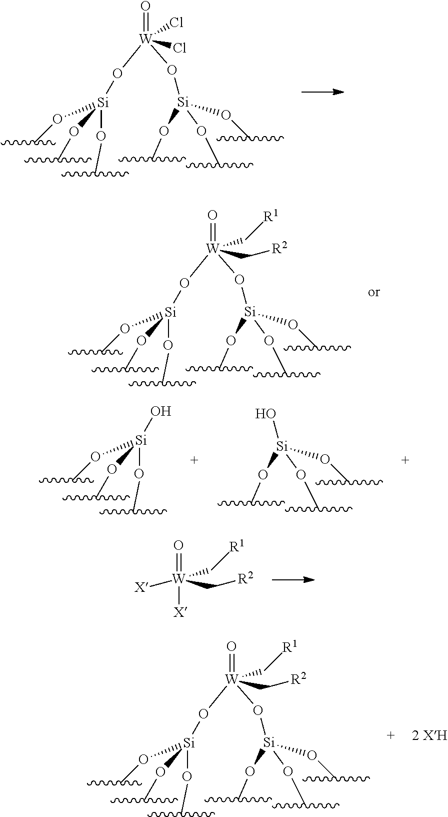

wherein

R¹ and R² have the same meaning as defined for the new catalyst of formula (III), being understood that R¹ and R² cannot be both hydrogen in formula (III),

X′ is chosen from chlorine, bromine, fluorine, aryloxy groups, siloxy groups or pyrolidyl groups, preferably X′ is selected from chlorine, bromine, fluorine or one of the following groups:

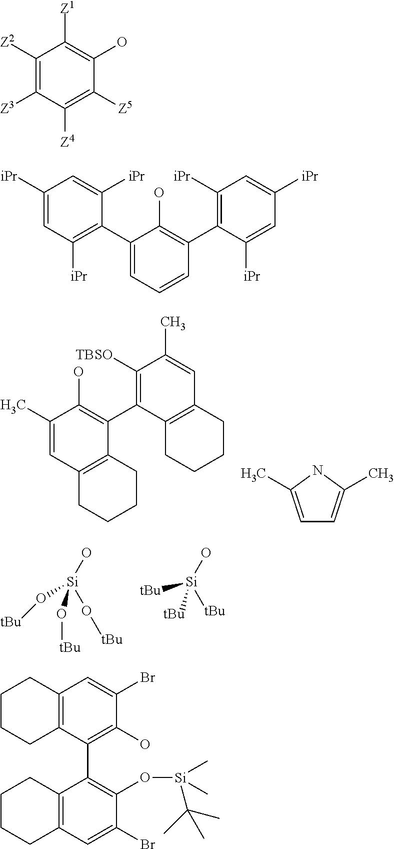

with Z¹, Z², Z³, Z⁴ and Z⁵ are independently to each other selected from hydrogen, methyl, tertio-butyl, adamantyl, mesityl, trifluoromethyl, fluoro, more preferably Z²═Z³═Z⁴═H and Z′ is identical to Z⁵ and is selected from methyl, tertio-butyl, adamantyl, mesityl.

The silica support may for example be dehydroxylated at a relatively low temperature (around 200° C.) before grafting the corresponding complex onto the silica support. The relatively low temperature for the dehydroxylation facilitates the formation of a bipodal catalyst. Compounds of formula (IVa) may be prepared according to a similar method as the method for preparing compounds of formula (IIIa), by replacing the tungsten atom by a molybdenum atom.

Compounds of formula (Va) may be prepared according to the following reaction:

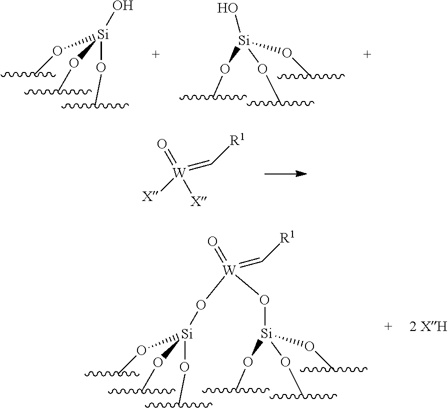

wherein

R¹ has the same meaning as defined for the catalyst of formula (V),

X″ is chosen from chlorine, bromine, fluorine, aryloxy groups, siloxy groups or pyrolidyl groups, preferably X″ is selected from chlorine, bromine, fluorine or one of the following groups:

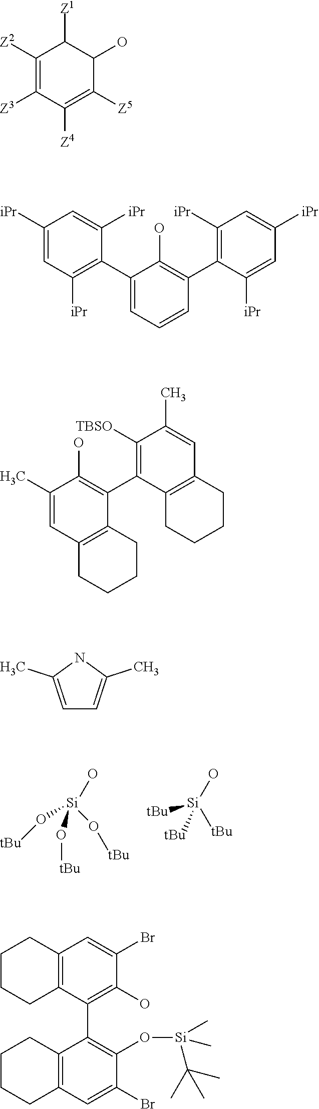

with Z¹, Z², Z³, Z⁴ and Z⁵ are independently to each other selected from hydrogen, methyl, tertio-butyl, adamantyl, mesityl, trifluoromethyl, fluoro, preferably Z²═Z³═Z⁴═H and Z′ is identical to Z⁵ and is selected from methyl, tertio-butyl, adamantyl, mesityl.

Compounds of formula (VIa) may be prepared according to a similar method as the method for preparing compounds of formula (Va), by replacing the tungsten atom by a molybdenum atom.

Compounds of formula (VIIa) may be prepared according to the following reaction:

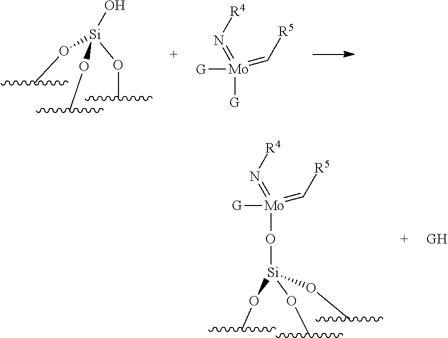

Compounds of formula (Villa) may be prepared according to the following reaction:

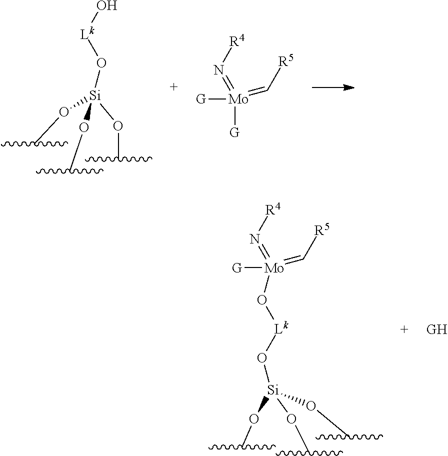

### EXAMPLES

### Example 1: Preparation and Characterization of Tungsten Oxo Catalyst Starting from a Tungsten Oxo Complex 1

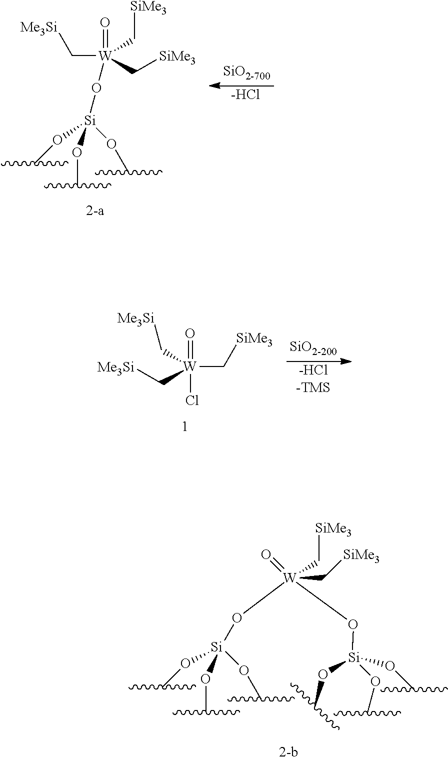

### Example 1a: Preparation of Monopodal Tungsten Oxo Catalyst 2-a

The grafting of 1 on silica dehydroxylated at 700° C. was thus performed under dynamic vacuum, to remove HCl and shift the equilibrium toward formation of the surface species. Infrared studies show the consumption of the isolated silanols at 3747 cm⁻¹. Furthermore, new peaks at 2850-3000 cm⁻¹ correspond to typically v(C—H) and δ(C—H) of alkyl fragments also appeared.

Elemental analysis of resulting material, 2-a, indicates a W and C % content of 4.54% wt and C 3.42% wt respectively. This corresponds to a C/W molar ratio of 14.4. In addition, the ¹H MAS and ¹³C CP MAS NMR data reveal the presence of tungsten methylenic fragments, as reflected by the ¹H and ¹³C signals at 1.3 and 66.23 ppm (FIG. 1). ²⁹Si MAS NMR spectrum exhibits two signals at 1.2 and −100 ppm, representing the neosilyl fragment and the silica support, respectively. From these combined spectroscopic and analytical elements, the reaction of 1 with the silica surface dehydroxylated at 700° C. by W-Cl silanolysis and concomitant HCl release leading to a monopodal surface species [(≡SiO)WONs₃] (2-a). This material is also characterized by XAFS. (Ns stands for neosilyl which corresponds to the group —CH₂SiMe₃).

### Example 1b Preparation of Bipodal Tungsten Oxo Catalyst 2-b

The grafting of 1 was performed under dynamic vacuum at 80° C. In order to prepare a well-defined bipodal supported tungsten derivative, we resorted to the use of moderate dehydroxylated silica (200° C.) that contains vicinal silanols. Complex 1 reacts readily with silica dehydroxylated at 200° C., to afford a yellow hybrid material. Infrared studies show quasi-quantitative consumption of the isolated silanols. Furthermore, new peaks correspond to typically v(C—H) of alkyl fragments also appeared. Elemental analysis indicates a W and C % content of 5.72% wt and C 3.15% wt respectively. This corresponds to a C/W molar ratio of 8.4. Thus, the characterization elements are in line with the formation of a major bipodal species [(SiO)₂WONs₂], 2-b. This catalyst is also characterized by XAFS and ²⁹Si NMR.

These types of bipodal catalysts can also be prepared by alkylation of bipodal oxo bis-chloride tungsten 3 by tetraneosilyltin according to the following schemes:

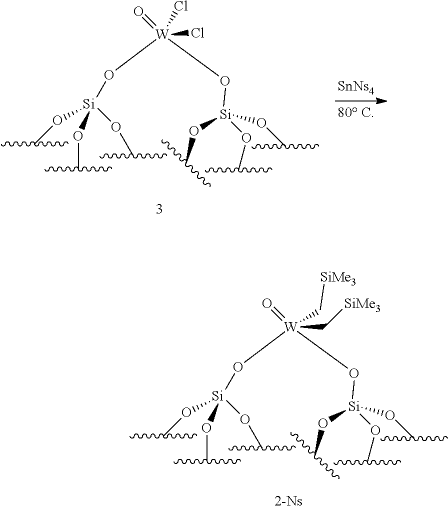

The bipodal oxo bis-chloride tungsten 3 may be prepared by grafting a WOCl₄ complex onto a silica support (SiO₂) dehydroxylated at 200° C. Said grafting may be performed according to a process similar to the process defined above (see example 1b).

### Example 2: Preparation of Oxo-Molybdenum Catalysts

### Example 2a: Preparation and Characterization of Monopodal Catalyst MoONp₃Cl/SiO₂₋₇₀₀

A mixture of finely ground MoONp₃Cl (120 mg, 0.33 mmol) and SiO₂₋₇₀₀ (1 g) were stirred at 25° C. under dynamic vacuum for 4 h, whereas all volatile compounds were condensed into a cold trap. Pentane was then added and the solid was washed 5 times. The resulting white powder was dried under vacuum (1×10⁻⁵ Torr). Analysis by infrared spectroscopy of the condensed volatiles indicated the formation of 218 μmol of HCl during the grafting (ca. 0.9 HCl/Mo). ¹H MAS NMR (500 MHz) δ2.6, 1.1 ppm. ¹³C CP MAS NMR (125 MHz) δ 86.3, 34.9, and 30.6 ppm. (Np stands for neopentyl which corresponds to the group —CH₂C(CH₃)₃).

### Example 2b: Preparation and Characterization of Bipodal Catalyst MoONp₃Cl/SiO₂₋₂₀₀

A mixture of finely ground MoONp₃Cl (130 mg, 0.375 mmol) and SiO₂₋₂₀₀ (1 g) were stirred at 25° C. under dynamic vacuum for 4 h, whereas all volatile compounds were condensed into a cold trap. Pentane was then added and the solid was washed 5 times. The resulting white powder was dried under vacuum (1×10⁻⁵ Torr) and the resulting solid was heated at 80° C. at 16 h. ¹H MAS NMR (500 MHz) δ2.6, 1.1 ppm. ¹³C CP MAS NMR (125 MHz) δ 86.7, 35.4, and 30.6 ppm. (Np stands for neopentyl which corresponds to the group —CH₂C(CH₃)₃).

### Example 3

Evaluation of the performances of the process of the invention Catalytic performance in methyl oleate conversion was studied in a stainless steel autoclave (60 mL autoclave in 7 mL of anhydrous and degassed toluene, unless it is otherwise mentioned) at different pressure, temperature and methyl oleate/W ratio. In a glove box toluene, appropriate amount of purified methyl oleate and optionally the scavenger (unless otherwise specified 200 mg of AliBu₃/SiO₂ are used in the catalytic tests) are gently mixed together before adding the catalyst. The autoclave is sealed then taken out from the glove box then tightened in the vice. The desired ethene (purified over adsorbents for O₂ and water removal) pressure is introduced in the autoclave then the reaction is heated at the desired temperature under stirring (200 rpm) (unless otherwise specified given pressures are initial pressure). After catalysis, the autoclave is cooled to room temperature in an ice bath then slowly depressurized. The walls are rinsed with a small volume of toluene (around 3 mL) and all the reaction mixture is transferred into a 20 mL vial. Around 400 mg (precisely weighed) of tetradecane is added as the external standard then the volume of the vial is completed to 20 mL with methanol. The mixture is homogenized then diluted 10 times in methanol. The diluted solution is injected in GC. The conversion and the selectivity were determined by online GC (HP 6890, equipped with 30 m HP5/Al₂O₃ column and an FID). The targeted products are 1-decene and methyl 9-decenoate.

Toluene is distilled over Na under argon flow, collected in a Rotaflo®, degassed by freeze thaw cycles then stored over activated molecular sieves in the glove box. The toluene is heated overnight at 100° C. over AliBu₃/SiO₂ (3 g/200 mL). After cooling and filtration of the solid, the toluene is stored in the glove box until its use.

General Procedure for AliBu₃/SiO₂ Scavenger Preparation:

Preparation of the Support:

Aerosil® 380 fumed silica (20 g) is compacted in distilled water (400 mL) then dried at 100° C. in the oven. The blocks are crushed then sieved to obtain Ø<450 μm particles. This silica is then dehydroxylated at 200° C. at atmospheric pressure. When no more water is condensing, the silica is dehydroxylated at 200° C. under high vacuum until the vacuum is lower than 5*10⁻⁵ mbar. The SiO_{2-380 D200} is stored under argon in a glove box until its use.

Functionalization of the Support:

In a glove box, SiO_{2-380 D200} is suspended in dry and degassed pentane (6 mL/g) then under gentle stirring AliBu₃ is slowly added (0.782 mmol/g). The reaction is gently stirred in the glove box at RT overnight then the solvent is removed under vacuum.

TON (Turn Over Number)=Conversion×(molar ratio=mol of methyl oleate/mol of W or Mo)

Conversion=mol of methyl oleate converted/mol of methyl oleate introduced×100 Selectivity in ethenolysis=[mol of 1-decene+mol of methyl 9-decenoate]/mol of reaction products×100. Reaction products comprise 1-decene and methyl 9-decenoate but also products from homometathesis reaction: 9-octadecene and dimethyl 9-octadecene-1,18-dioate as well as isomerization products of for example 1-decene and methyl 9-decenoate.

The ethenolysis of methyl oleate is represented by the following reaction:

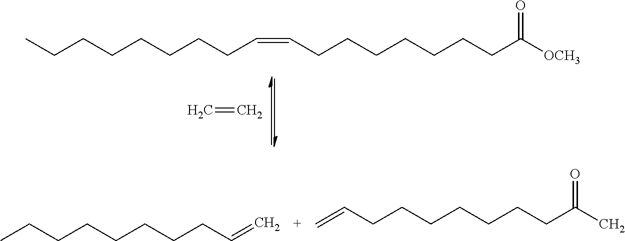

### Example 3a: Monopodal Catalyst 2-a

Catalyst 2-a was evaluated in the following conditions in the ethenolysis of methyl oleate:
Methyl oleate/W molar ratio=100;
Temperature=100° C.;
Initial Pressure=10 bar;
AliBu₃/SiO₂=200 mg.

TABLE 1
Conversions, selectivity and TON of Example 4a

| Time (h) | Conversion % | Selectivity % | TON |
|---|---|---|---|
| 1 | 4 | 98 | 4 |

We observe a relatively low conversion at one hour but with a very high selectivity. High conversion rates could be obtained by optimizing the operating conditions, such as reaction time or temperature.

### Example 3b: Bipodal Catalyst 2-b

Catalyst 2-b was evaluated in the following conditions in the ethenolysis of methyl oleate:
methyl oleate/W molar ratio=100 or 1000;
Temperature=100° C.;
Initial pressure=10 bar;
AliBu₃/SiO₂=200 mg.

TABLE 2
Conversions, selectivity and TON of Example
4b with a methyl oleate/W ratio of 100
Methyl oleate/W = 100

| Time (h) | Conversion % | Selectivity % | TON |
|---|---|---|---|
| 1 | 65 | 97 | 65 |
| 3 | 74 | 96 | 74 |
| 5 | 83 | 97 | 83 |

TABLE 2bis
Conversions, selectivity and TON of Example
3b with a methyl oleate/W ratio of 1000
Methyl oleate/W = 1000

| Time (h) | Conversion % | Selectivity % | TON |
|---|---|---|---|
| 1 | 14 | 80 | 140 |
| 3 | 20 | 86 | 200 |
| 5 | 33 | 95 | 330 |

With both ratios, we observe a very high selectivity (98%) and we observe that the conversion is better when the methyl oleate/W ratio is 100.

### Example 3c: Bipodal Catalyst 2-b

Catalyst 2-b was evaluated in the following conditions in the ethenolysis of methyl oleate:
methyl oleate/W molar ratio=100 or 1000;
Temperature=150° C.;
Initial pressure=10 bar;
AliBu₃/SiO₂=200 mg.

TABLE 3
Conversions, selectivity and TON of Example 3c

| Methyl oleate/W | Time (h) | Conversion % | Selectivity % | TON |
|---|---|---|---|---|
| 100 | 1 | 89 | 95 | 89 |
| 1000 | 1 | 43 | 95 | 470 |

We observe that the conversion is higher when the temperature increases from 100° C. to 150° C. The selectivity is still very high, 95% and 94%.

### Example 3d: Bipodal Catalyst 2-Ns

Catalyst 2-Ns was evaluated in the following conditions in the ethenolysis of methyl oleate:
Methyl oleate/W molar ratio=100;
Temperature=100° C.;
Initial pressure=10 bar.

TABLE 4
Conversions, selectivity and TON of Example 3d

| Time (h) | Conversion % | Selectivity % | TON |
|---|---|---|---|
| 1 | 21 | 98 | 21 |

We observe a very high selectivity (98%).

### Conclusion of Example 3

Comparison of the tested catalysts at reaction time 1 h, 100° C., 10 bar and oleate/W molar ratio of 100:

TABLE 5
Conclusion on the different tested catalysts

| | Catalyst 2-b | Catalyst 2-a | Catalyst 2-Ns |
|---|---|---|---|
| | bipodal | monopodal | bipodal |
| | (example 2b) | (example 2a) | (example 2d) |
| Conversion % | 65 | 4 | 21 |
| Selectivity % | 97 | 98 | 98 |
| TON | 65 | 4 | 21 |

As shown in the above-table 5, all the catalysts tested show a selectivity of more than 90% (94% and 98%).

The catalyst 2-b bipodal presents a higher conversion and selectivity than the other tested catalysts. In particular, we can note that the catalyst 2-b obtained by grafting the corresponding complex onto the support gives a higher conversion than the catalyst 2-Ns obtained by reacting a bipodal oxo bis-chloride tungsten with SnNs₄.

### Example 4—Evaluation of the Process with Different Conditions

A similar ethenolysis reaction process as the one of example 3 was performed with the catalyst 2-b defined above, at different experimental conditions, such as temperatures, pressure, oleate/W molar ratio.

### Example 4a: Evaluation of the Influence of the Pressure

### Example 4a-1: Catalyst 2-b was Evaluated in the Following Conditions in the Ethenolysis of Methyl Oleate

methyl oleate/W molar ratio=100;
Temperature=100° C.;
Initial pressure=10 bar, 20 bar or 40 bar.

TABLE 6
Conversions, selectivity and TON of Example 4a-1

| Time (h) | Initial Pressure (bar) | Conversion % | Selectivity % | TON |
|---|---|---|---|---|
| 1 | 10 | 65 | 97 | 65 |
| 1 | 20 | 51 | 90 | 51 |
| 1 | 40 | 37 | 90 | 37 |

We observe in table 6 above that the selectivity is high at 10 bar, 20 bar and 40 bar. We can note that the conversion decreases when the pressure increases.

### Example 4a-2

Catalyst 2-b was evaluated in the following conditions in the ethenolysis of methyl oleate:
methyl oleate/W molar ratio=1000;
Temperature=100° C.;
Constant pressure=0.5 bar, 1 bar, 2 bar, 5 bar and 10 bar.

TABLE 7
Conversion, selectivity and Ton of example 4a-2

| | Constant Pressure | | | |
|---|---|---|---|---|
| Time (h) | P(C₂H₄) | Conversion (%) | Selectivity (%) | TON |
| 1 | 0.5 | 10 | 82 | 100 |
| 3 | 0.5 | 22 | 75 | 220 |
| 15 | 0.5 | 32 | 61 | 320 |
| 1 | 1 | 9 | 76 | 90 |
| 3 | 1 | 22 | 90 | 220 |
| 15 | 1 | 42 | 93 | 420 |
| 1 | 2 | 14 | 91 | 140 |
| 3 | 2 | 22 | 89 | 220 |
| 15 | 2 | 45 | 94 | 450 |
| 1 | 5 | 17 | 93 | 170 |
| 3 | 5 | 28 | 96 | 280 |
| 15 | 5 | 47 | 97 | 470 |
| 1 | 10 | 8 | 90 | 80 |
| 3 | 10 | 15 | 93 | 150 |
| 15 | 10 | 35 | 97 | 250 |

We observe that the final conversion is similar for 1, 2 and 5 bars but decreases at 0.5 and 10 bars, and that selectivity in cross-metathesis is higher for P(C2H4) higher than 1 bar.

### Example 4b: Evaluation of the Influence of the Temperature

### Example 4b-1: Catalyst 2-b was Evaluated in the Following Conditions in the Ethenolysis of Methyl Oleate

methyl oleate/W molar ratio=100;
Temperature=100° C., 150° C. or 200° C.;
Initial pressure=10 bar.

TABLE 8
Conversions, selectivity and TON of Example 4b-1

| Time (h) | Temperature (° C.) | Conversion % | Selectivity % | TON |
|---|---|---|---|---|
| 1 | 100 | 65 | 97 | 65 |
| 1 | 150 | 89 | 95 | 89 |
| 1 | 200 | 93 | 95 | 93 |

We observe that the selectivity is high at 100° C., 150° C. and 200° C. We can note that the conversion increases when the temperature increases.

### Example 4b-2: Catalyst 2-b was Evaluated in the Following Conditions in the Ethenolysis of Methyl Oleate

methyl oleate/W molar ratio=1000;
Temperature=120° C. or 150° C.;
Constant pressure=5 bar.

TABLE 9
Conversion, selectivity and TON of example 4b-2

| Temperature (° C.) | Time (h) | Conversion (%) | Selectivity (%) | TON |
|---|---|---|---|---|
| 120 | 1 | 29 | 94 | 290 |
| 120 | 3 | 38 | 95 | 380 |
| 120 | 15 | 50 | 94 | 500 |
| 150 | 1 | 36 | 94 | 360 |
| 150 | 3 | 51 | 93 | 510 |
| 150 | 5 | 57 | 94 | 570 |

We observe that the selectivity is high for different times of reaction and different temperatures.

### Example 4c: Evaluation of the Influence of the Methyl Oleate/W Ratio

Catalyst 2-b was evaluated in the following conditions in the ethenolysis of methyl oleate:
methyl oleate/W molar ratio=100, 500 or 1000;
Temperature=100° C.;
Initial pressure=10 bar.

TABLE 10
Conversions, selectivity and TON of Example 4c

| | Methyl oleate/W | | | |
|---|---|---|---|---|
| Time (h) | molar ratio | Conversion % | Selectivity % | TON |
| 1 | 100 | 65 | 97 | 65 |
| 1 | 500 | 17 | 90 | 85 |
| 1 | 1000 | 14 | 80 | 140 |
| 1 | 1000* | 17 | 90 | 170 |

*This test were performed in the presence of 500 mg of Al(iBu)₃/SiO₂.

We observe in table 10 above that the selectivity is better when the methyl oleate/W ratio is of 100 and 500. We also note that the conversion is better when the methyl oleate/W ratio is of 100.

We also observe that the scavenger allows improving the selectivity of the reaction.

### Example 5—Tests with Another Oxo-W Based Catalyst

Another oxo-W based catalyst was prepared according to the following scheme and process:

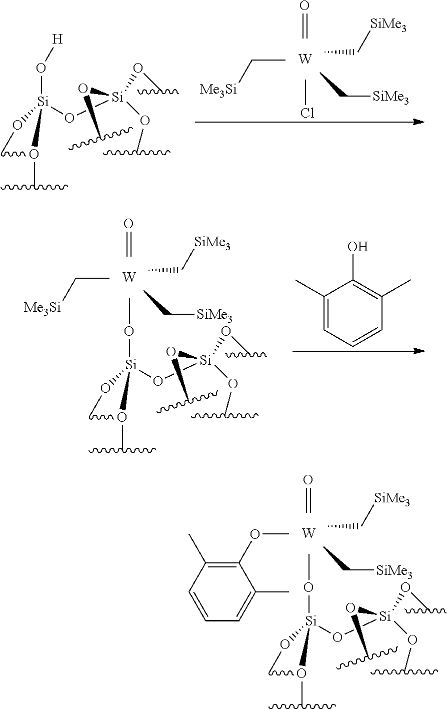

A mixture of finely ground [WO(CH₂SiMe₃)₃Cl] (175 mg, 0.351 mmol) and SiO₂₋₇₀₀ (1 g) was stirred at 40° C. (5 h) under dynamic vacuum whilst all volatile compounds were condensed into a cold trap. Pentane was then added and the solid was washed 5 times. The resulting white powder was dried under vacuum (10⁻⁵ Torr). The latter materials was further reacted with 2,6-dimethyl phenol in heptane at 80° C. for 12 hours. The product was obtained after extensive washing with heptane and dried under high vacuum.

A catalytic test of the WO(Ns)₃/SiO₂₋₇₀₀ modified using 2,6-dimethylphenol (WO(OAr)(Ns)₂/SiO₂₋₇₀₀) has been performed according to the ethenolysis process defined in example 3 with the following conditions: ratio methyl oleate/W=1000, 100° C.; 10 bar for 15 h.

TABLE 11
Comparison of catalytic activity of different W based
catalysts (methyl oleate/W = 1000; T° = 100°
C.; initial P_{C2H4} = 10 bar; m_{AliBu3/SiO2} = 200 mg; t = 15 h).

| Catalyst | Conversion (%) | Selectivity (%) | TON |
|---|---|---|---|
| WO(Ns)₂/SiO₂₋₂₀₀ (2-b) | 52 | 95 | 520 |
| WO(OAr)(Ns)₂/SiO₂₋₇₀₀ | 22 | 94 | 220 |

The catalyst WO(OAr)(Ns)₂/SiO₂₋₇₀₀ provides a satisfying selectivity, even if we can observe that the catalyst WO(Ns)₂/SiO₂₋₂₀₀ provides in those conditions a higher conversion.

### Example 6—Evaluation of Imido-Mo Catalysts

The following imido-Mo based catalysts have been prepared and evaluated:

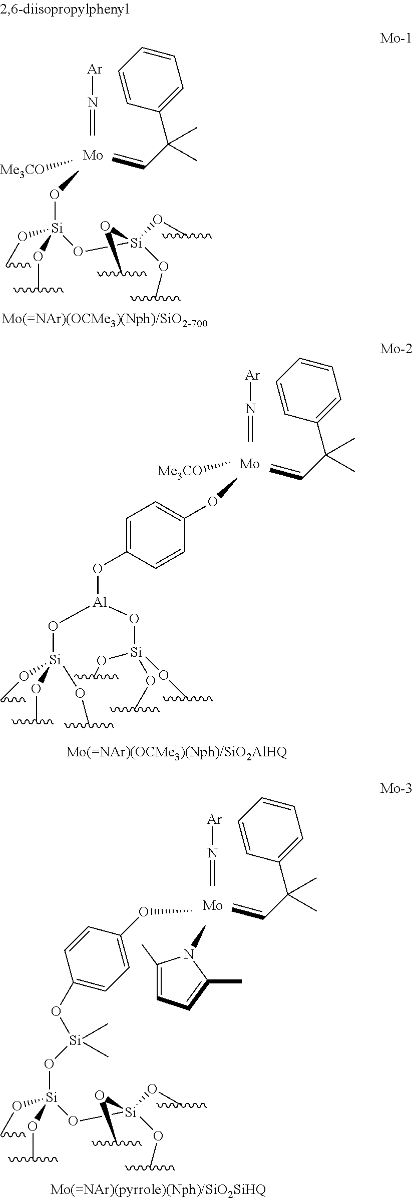

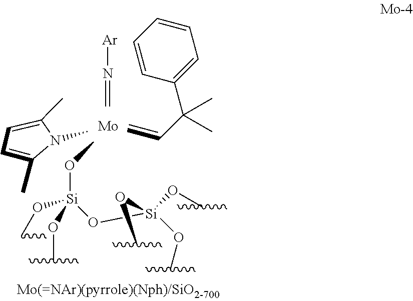

Mo-1 Catalyst has been Prepared According to the Following Scheme and Process:

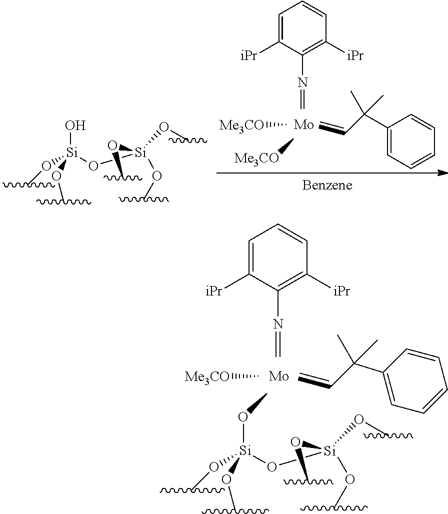

An excess of the commercial complex Mo(C₁₀H₁₂)(C₁₂H₁₇N)(OC₄H₉)₂ (280 mg) is dissolved in dry benzene and reacted with silica dehydroxylated at 700° C. (1 g) for 2 hours at room temperature. The product was isolated after extensive washing with benzene and dried under high vacuum. Elemental analysis: Mo=2.22 wt %; C=6.8 wt %; N=0.58 wt %.

Mo-2 Catalyst has been Prepared According to the Following Scheme and Process:

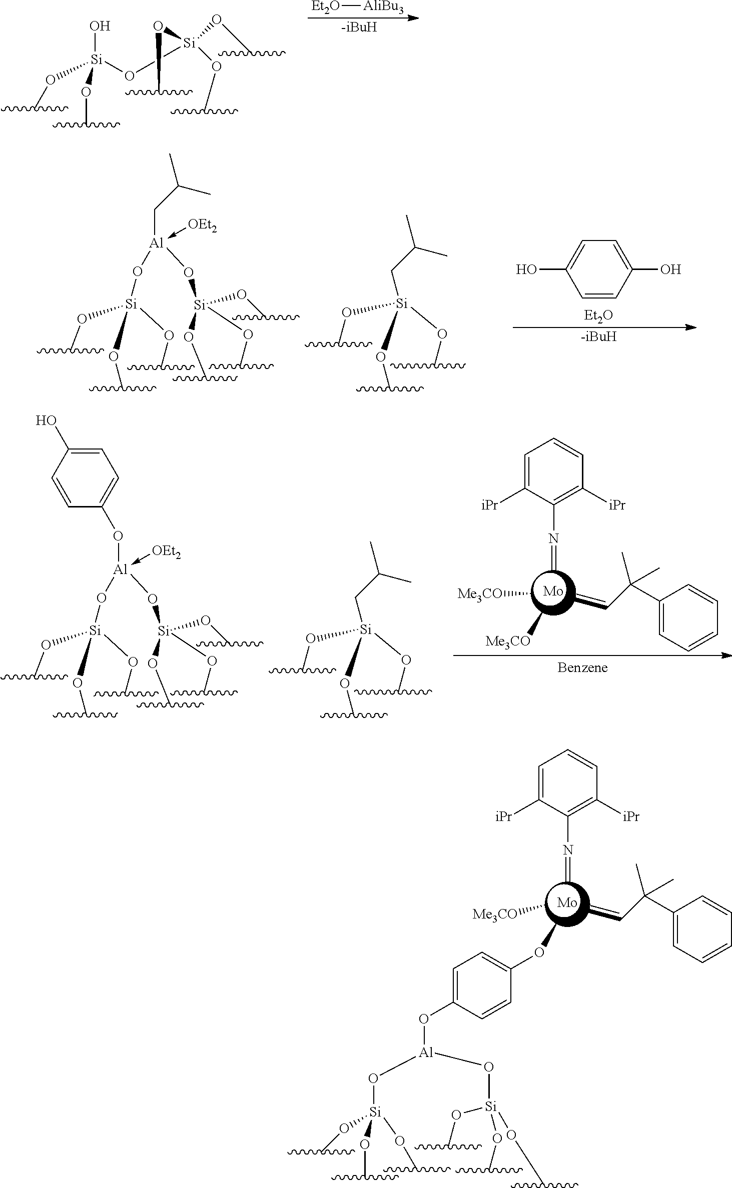

3 g of SiO₂₋₇₀₀ were contacted with a solution of AliBu₃ (0.3 ml) in diethylether, and stirred at 25° C. overnight in a double-Schlenck. After filtration, the solid was washed three times with diethylether. The resulting white powder was dried under vacuum. 2 g of the latter material was contacted with a solution of hydroquinone (90 mg) in diethylether, and stirred at 25° C. overnight in a double-Schlenck. After filtration, the solid was washed three times with diethylether. The resulting white powder was dried under high vacuum. Then, the commercial complex Mo(C₁₀H₁₂)(C₁₂H₁₇N)(OC₄H₉)₂ (280 mg) is dissolved in dry benzene and reacted with the functionalized silica (1 g) at room temperature for 2 hours. The product was isolated after extensive washing with benzene and dried under high vacuum. Elemental analysis: Mo=2.04 wt %; C=8.67 wt %; N=0.44 wt %.

Mo-3 Catalyst has been Prepared According to the Following Scheme and Process:

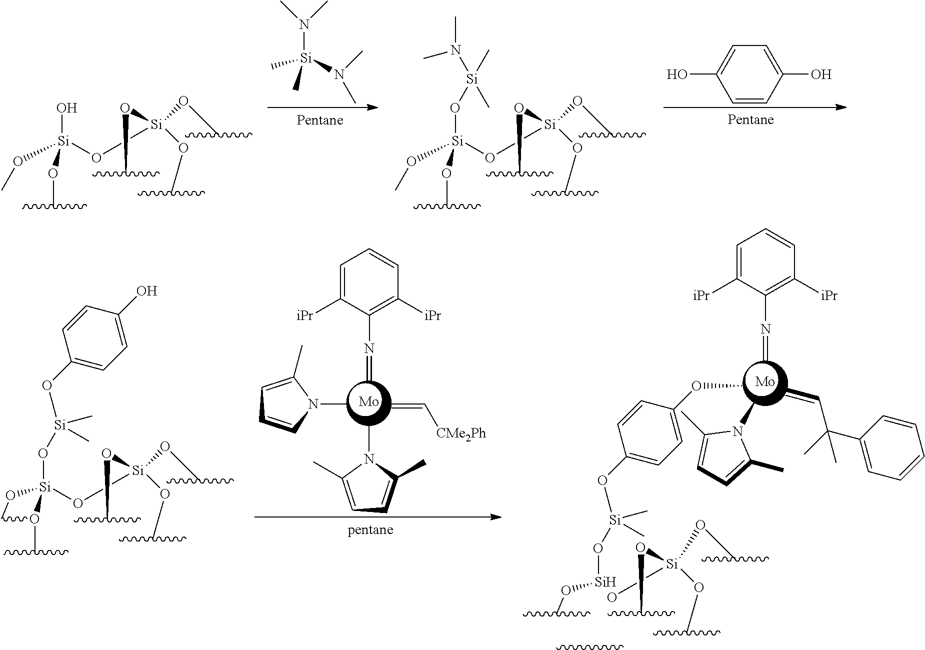

3 g of SiO₂₋₇₀₀ were contacted with a solution of bis(dimethylamino)dimethyl silane (0.2 ml) in pentane, and stirred at 25° C. for 3 hours in a double-Schlenck. After filtration, the solid was washed three times with pentane. The resulting white powder was dried under vacuum. 2 g of the latter material was contacted with a solution of hydroquinone (90 mg) in ether, and stirred at 25° C. overnight in a double-Schlenck. After filtration, the solid was washed three times with diethylether. The resulting white powder was dried under high vacuum. Then, Mo(CHCMe₂Ph) (C₁₂H₁₇N) (2,5-Me₂-NC₄H₂)₂ (300 mg; prepared according to Organometallics 2007, 26, 2528) is dissolved in dry pentane and reacted with the functionalized silica (1 g) at room temperature for 2 hours. The product was isolated after extensive washing with benzene and dried under high vacuum. Elemental analysis: Mo=1.44 wt %; C=7.74 wt %; N=0.42 wt %.

Mo-4 Catalyst has been Prepared According to the Following Scheme and Process:

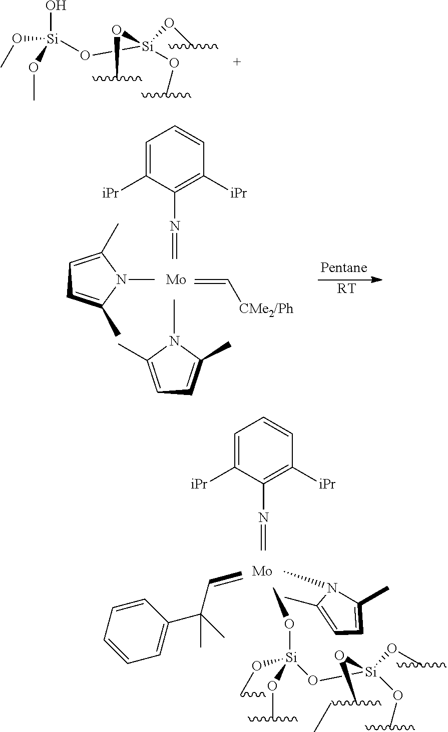

An excess of Mo(CHCMe₂Ph) (C₁₂H₁₇N) (2,5-Me₂-NC₄H₂)₂ (300 mg; prepared according to Organometallics 2007, 26, 2528) is dissolved in dry pentane and reacted with silica dehydroxylated at 700° C. (1 g) for 2 hours at room temperature (RT). The product was isolated after extensive washing with pentane and dried under high vacuum. Elemental analysis: Mo=2.22 wt %; C=5.33 wt %.

### Example 6a: Evaluation of Imido-Mo Catalysts

Catalytic tests have been performed according to the ethenolysis process defined in example 3 with the following conditions: a molar ratio methyl oleate/Mo=1000; T=100° C., initial P_{C2H4}=10 bar; m_{AliBu3/SiO2}=200 mg for 1 h (in the presence of scavenger).

TABLE 12
Evaluation of imido-molybdenum catalysts

| Catalysts | Conversion (%) | Selectivity (%) | TON |
|---|---|---|---|
| Mo-1 | 13 | 83 | 130 |
| Mo-2 | 8 | 72 | 80 |
| Mo-3 | 36 | 51 | 360 |

Results that are presented in the table 12 show that catalysts Mo-1 & Mo-3 give the best conversion. Mo-1 catalyst is very selective in ethenolysis products (with a selectivity of 83%).

### Example 6b: Imido-Mo Pyrrole Catalysts (Mo-3 Catalyst)

The conversion and selectivity were measured after 1 h at 100° C. and molar ratio methyl oleate/Mo=1000 under initial ethene pressures from 2 to 20 bar. The results are presented in the table 13.

TABLE 13
Effect of initial ethene pressure on the methyl oleate
ethenolysis with Mo-3 catalyst (methyl oleate/Mo = 1000;
T° = 100° C.; m_{AliBu3/SiO2} = 200 mg; t = 1 h).

| P_{C2H4} (bar) | Conversion (%) | Selectivity (%) | TON |
|---|---|---|---|
| 2 | 50 | 71 | 500 |
| 5 | 38 | 66 | 380 |
| 10 | 36 | 51 | 360 |
| 20 | 34 | 41 | 340 |

As illustrated in table 13, one can see that decreasing the pressure allows improving the conversion and the selectivity. The effect of the time of reaction has also been evaluated at an initial ethene pressure of 2 bar. We observed in table 14 that the conversion increased very fast in the first hour to reach a plateau around 50%. We also observed that the good selectivity around 70% obtained at 1 h decreased and stabilized around 50% with time. This observation is probably due to the decrease of ethene concentration in the solution.

TABLE 14
Kinetics & selectivity of methyl oleate ethenolysis with
Mo-3 catalyst at 100° C. (methyl oleate/Mo = 1000; T° =
100° C.; initial P_{C2H4} = 2 bar; m_{AliBu3/SiO2} = 200 mg).

| Time (h) | Conversion (%) | Selectivity (%) | TON |
|---|---|---|---|
| 1 | 50 | 71 | 500 |
| 3 | 47 | 55 | 470 |
| 15 | 49 | 55 | 490 |

The effect of temperature has also been evaluated at an initial ethene pressure of 2 bar. Therefore, the same test has been performed at a temperature of 50° C. (instead of 100° C.). We observed in table 15 that the conversion increased very fast in the first hour to reach a plateau around 40% that is a very similar to the result obtained at 100° C. A decrease of the temperature from 100° C. to 50° C. appeared to have only a small influence on the methyl oleate conversion. The selectivity in cross-metathesis products appeared to be also not influenced by this temperature change.

TABLE 15
Kinetics & selectivity of methyl oleate ethenolysis with
Mo-3 catalyst at 50° C. (methyl oleate/Mo = 1000; T° =
50° C.; initial P_{C2H4} = 2 bar; m_{AliBu3/SiO2} = 200 mg).

| Time (h) | Conversion (%) | Selectivity (%) | TON |
|---|---|---|---|
| 1 | 37 | 56 | 370 |
| 3 | 41 | 55 | 410 |
| 15 | 38 | 60 | 380 |

### Example 6c: Evaluation of Mo-4 Catalyst

The only difference concerning the active site in Mo-4 catalyst from the one in Mo-3 catalyst is the aryloxylinker that keep away the metal from the silica surface in the latter. The ethenolysis reaction has been performed with Mo-4 catalyst according to the same process as described in example 3.

At 50° C., the conversion obtained using the Mo-4 catalyst is significantly higher than the one obtained with Mo-3 catalyst. The support does not have negative effect on the methyl oleate conversion but we observe that Mo-4 catalyst provides an improved selectivity for ethenolysis products, as compared with Mo-3 catalyst. Kinetics of methyl oleate ethenolysis with Mo-4 were also performed at 100° C. under initial low ethene pressure P_{C2H4}=2 bar (table 16).

TABLE 16
Kinetics & selectivity of methyl oleate ethenolysis with
Mo-4 catalyst at 50 & 100° C. (methyl oleate/Mo = 1000; initial
P_{C2H4} = 2 bar; m_{AliBu3/SiO2} = 200 mg).

| T(° C.) | Time (h) | Conversion (%) | Selectivity (%) | TON |
|---|---|---|---|---|
| 100 | 1 | 55 | 89 | 550 |
| | 3 | 55 | 88 | 550 |
| 50 | 1 | 49 | 94 | 490 |
| | 3 | 57 | 94 | 570 |

The evolutions of conversions with time at 50 & 100° C. are very similar reaching in 1 h a plateau around 55%.

### Example 6d—Imido-Molybdenum-Tbutoxy Catalysts (Mo-1 Catalyst)

Kinetics of methyl oleate ethenolysis with Mo-1 were performed at 50 & 100° C. under constant low ethene pressure P_{C2H4}=5 bar. Similarly to what has been observed with Mo-3 & Mo-4 catalysts 50 & 100° C., the methyl oleate conversion with Mo-1 catalyst is faster at the beginning of the reaction. The conversion is slowly increasing from 9 to 18% at 50° C. and from 20 to 32% at 100° C. (table 17).

TABLE 17
Kinetics & selectivities of methyl oleate ethenolysis
with Mo-1 catalyst at 50 & 100° C. (methyl oleate/Mo =
1000; P_{C2H4} = 5 bar constant; m_{AliBu3/SiO2} = 200 mg).

| T (° C.) | Time (h) | Conversion (%) | Selectivity (%) | TON |
|---|---|---|---|---|
| 100 | 1 | 20 | 96 | 200 |
| | 3 | 22 | 97 | 220 |
| | 15 | 32 | 97 | 320 |
| 50 | 1 | 9 | 93 | 90 |
| | 3 | 11 | 95 | 110 |
| | 15 | 18 | 97 | 180 |

Concerning the selectivity, performing the ethenolysis at 5 bar (constant) allowed to increase the selectivity in cross-metathesis products that we obtained at 10 bar (83%) to values higher than 95%.

1. A process for obtaining alpha-olefins, said process comprising a step of reacting optionally-functionalized internal unsaturated olefins with ethylene in the presence of a supported catalyst selected from a supported oxo-molybdenum or imido-molybdenum catalyst or a supported oxo-tungsten catalyst,
said oxo-tungsten catalyst being selected from one of the following oxo-tungsten compounds:
□-W(═O)X(CH₂R¹)(CH₂R²) (I)
(□)₂W(═O)(CH₂R¹)(CH₂R²) (III)
said imido-molybdenum catalyst being selected from one of the following imido-molybdenum compounds:
□-OL^{k}O—Mo(═NR⁴)G(═CHR⁵) (VIII)
wherein,
□ corresponds to a support,
R¹ and R², are independently to each other, selected from hydrogen, linear or branched alkyl groups, —C(CH₃)₃, -Phenyl, —Si(CH₃)₃, —C(CH₃)₂Ph, being understood that R¹ and R² cannot be both hydrogen in formula (III),
X is selected from alkoxy groups, aryloxy groups, —Si(CH₃)₃, siloxy groups or pyrolidyl groups,
R⁴ represents a radical selected from aliphatic and aromatic hydrocarbyl radicals, optionally comprising one or more heteroatoms,
R⁵ is selected from hydrogen, linear or branched alkyl groups, —C(CH₃)₃, -Phenyl (Ph), —Si(CH₃)₃, or —C(CH₃)₂Ph,
G is selected from alkoxy groups, aryloxy groups, siloxy groups or pyrolidyl groups,
L^{k} represents a divalent linker.

2. The process according to claim 1, wherein the optionally-functionalized internal unsaturated olefins are selected from optionally-functionalized internal mono-unsaturated olefins.

3. The process according to claim 1, wherein:
R¹, R² and R⁵, are independently to each other, selected from —H, methyl, ethyl, propyl, isopropyl, butyl, isobutyl, pentyl, isopentyl, n-hexyl, —C(CH₃)₃, -Phenyl, —Si(CH₃)₃, or —C(CH₃)₂Ph, and/or
X and G are selected from the following groups: 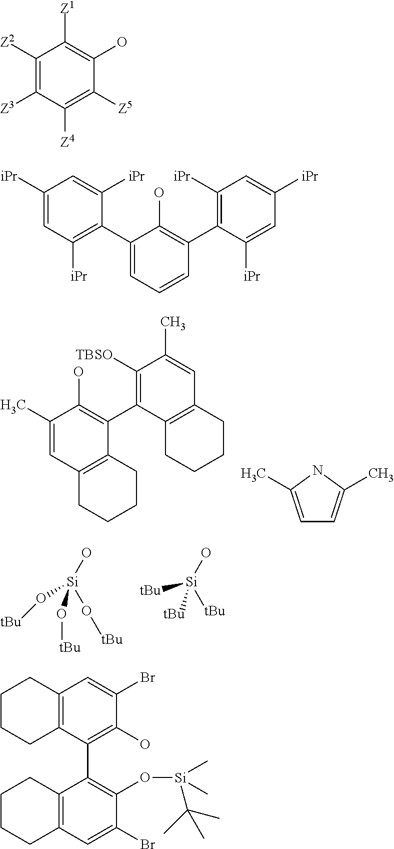
or the radical —O—C(R⁶)₃,
with
Z¹, Z², Z³, Z⁴ and Z⁵ are independently to each other selected from hydrogen, methyl, tertio-butyl, adamantyl, mesityl, trifluoromethyl, fluoro more preferably
Z²═Z³═Z⁴═H and Z⁵ is identical to Z⁵ and is selected from methyl, tertio-butyl, adamantyl, mesityl,
R⁶ is a linear, branched or cyclic alkyl radical having preferably from 1 to 12 carbon atoms.

4. The process according to claim 1, wherein the optionally-functionalized internal unsaturated olefins comprise from 8 to 50 carbon atoms.

5. The process according to claim 1, wherein the optionally-functionalized internal unsaturated olefins are functionalized by at least one functional group in terminal position of the olefin.

6. The process according to claim 5, wherein the functional group is chosen from ester, acid, amide, amine, alcohol.

7. The process according to claim 1, wherein the optionally-functionalized internal unsaturated olefins are chosen from alkyl oleate.

8. The process according to claim 1, wherein the optionally-functionalized internal unsaturated olefins are methyl oleate compounds and the alpha-olefins are 1-decene compounds.

9. The process according to claim 1, wherein the support of the catalyst is chosen from silica, modified silica, alumina, modified alumina, titanium oxide, niobium oxide, silica-alumina, organic polymers, and polystyrene beads.

10. The process according to claim 1, wherein oxo-molybdenum catalyst does not comprise any carbene function.

11. The process according to claim 1, wherein the oxo-molybdenum catalyst is a monopodal or a bipodal catalyst.

12. The process according to claim 1, wherein the supported catalyst is selected from:
the compounds of formula (I): □-W(═O)X(CH₂R¹)(CH₂R²), preferably of formula (Ia): 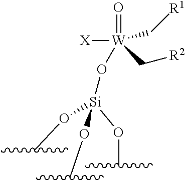
the compounds of formula (II): □-Mo(═O)X(CH₂R¹)(CH₂R²), preferably of formula (IIa): 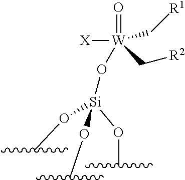
the compounds of formula (III): (□)₂W(═O) (CH₂R¹)(CH₂R²); preferably of formula (IIIa): 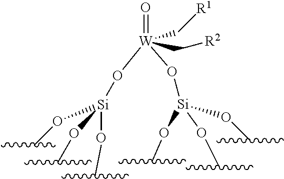
the compounds of formula (IV): (□)₂Mo(═O) (CH₂R¹)(CH₂R²); preferably of formula (IVa): 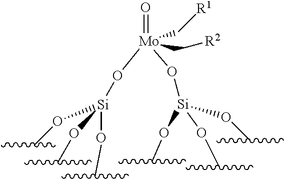
the compounds of formula (VI): (□)₂Mo(═O)(═CHR⁵); preferably of formula (VIa): 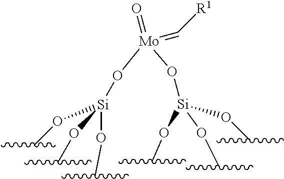
the compounds of formula (VII): □-Mo(═NR⁴)G(═CHR⁵); preferably of formula (VIIa): 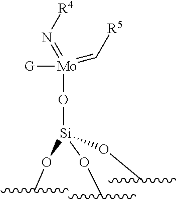
the compounds of formula (VIII): □-OL^{k}O—Mo(═NR⁴)G(═CHR⁵); preferably of formula (VIIIa): 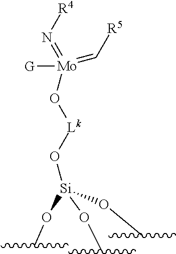
preferably the supported catalyst is selected from the compounds of formula (I), preferably (Ia), of formula (II), preferably (IIa), of formula (III), preferably (IIIa) or of formula (IV), preferably (IVa).

13. The process according to claim 12, wherein the supported catalyst is a compound of formula (III), or a compound of formula (IV).

14. The process according to claim 1, wherein the catalyst is obtained by grafting the corresponding complex onto the support □.

15. The process according to claim 1, wherein the reaction is performed at a temperature ranging from 0° C. to 400° C.

16. The process according to claim 1, wherein the reaction is performed at a pressure ranging from 1 to 300 bar.

17. The process according to claim 1, wherein the functionalized internal olefins have a purity of at least 99%.

18. The process according to claim 1, wherein at the beginning of the reaction, the optionally-functionalized internal unsaturated olefins/(W or Mo) molar ratio ranges from 50 to 5000.

19. The process according to claim 1, comprising, before the step of reacting, a step of the purification of optionally-functionalized internal unsaturated olefins.

20. The process according to claim 1, wherein the reaction is performed in the presence of a scavenger.

21. A supported catalyst selected from a supported oxo-molybdenum catalyst or a supported oxo-tungsten catalyst or a supported imido-molybdenum catalyst responding to the following formula:
□-W(═O)X(CH₂R¹)(CH₂R²) (I)
□-Mo(═O)X(CH₂R¹)(CH₂R²) (II)
(□)₂W(═O)(CH₂R¹)(CH₂R²) (III)
(□)₂Mo(═O)(CH₂R¹)(CH₂R²) (IV)
(□)₂Mo(═O)(═CHR¹) (VI)
□-Mo(═NR⁴)G(═CHR⁵) (VII)
□-OL^{k}O—Mo(═NR⁴)G(═CHR⁵) (VIII)
wherein,
□ corresponds to a support,
R¹ and R², are independently to each other, selected from hydrogen, linear or branched alkyl groups, the alkyl group preferably having from 1 to 12 carbon atoms, —C(CH₃)₃, -Phenyl, —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Phenyl, —Si(CH₃)₃, —C(CH₃)₂Ph,
being understood that R¹ and R² cannot be both hydrogen in formula (III),
R⁴ represents a radical selected from aliphatic and aromatic hydrocarbyl radicals, optionally comprising one or more heteroatoms, preferably comprising from 1 to 36 carbon atoms, preferably from 2 to 28 carbon atoms, more preferably from 3 to 24 carbon atoms,
R⁵ is selected from hydrogen, linear or branched alkyl groups, —C(CH₃)₃, -Phenyl (Ph), —Si(CH₃)₃, or —C(CH₃)₂Ph,
G is selected from alkoxy groups, aryloxy groups, siloxy groups or pyrolidyl groups,
L^{k} represents a divalent linker, preferably chosen from a linear, branched or cyclic alkylene, having preferably from 1 to 12 carbon atoms, or an arylene group optionally substituted having preferably from 6 to 12 carbon atoms,
X is selected from aryloxy groups, —Si(CH₃)₃, siloxy groups or pyrolidyl groups,
preferably X and G are selected from the following groups: 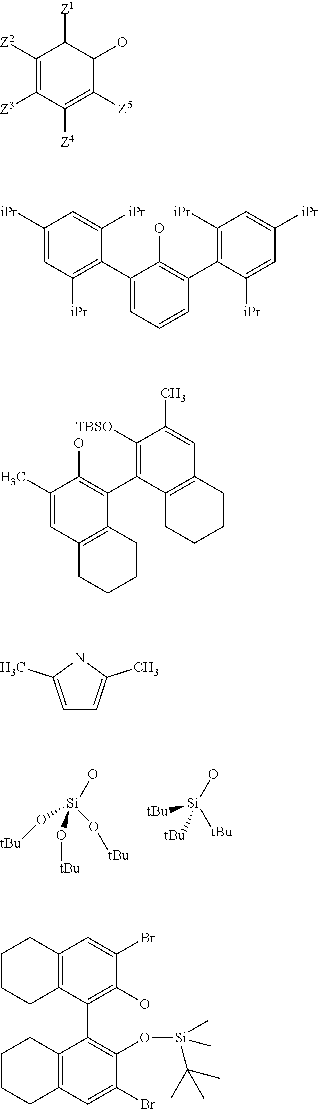
or the radical —O—C(R⁶)₃,
with
Z¹, Z², Z³, Z⁴ and Z⁵ are independently to each other selected from hydrogen, methyl, tertio-butyl, adamantyl, mesityl, trifluoromethyl, fluoro, preferably Z²═Z³═Z⁴═H and Z¹ is identical to Z⁵ and is selected from methyl, tertio-butyl, adamantyl, mesityl, and
R⁶ is a linear, branched or cyclic alkyl radical having preferably from 1 to 12 carbon atoms.

22. A method for preparing the supported catalyst of formulas (I), (II), (Ill), (IV), (VI), (VII) and (VIII), the method comprising one of the following reaction schemes:
(a) Reaction scheme 1 for obtaining catalysts of formula (I):
□-OH+W(═O)X(CH₂R¹)(CH₂R²)(CH₂R³)→□-W(═O)X(CH₂R¹)(CH₂R²)
(b) Reaction scheme 1 bis for obtaining catalysts of formula (I):
□-OH+W(═O)(CH₂R¹)(CH₂R²)(CH₂R³)→□-W(═O)(CH₂R¹)(CH₂R²)(CH₂R³)
□-W(═O)(CH₂R¹)(CH₂R²)(CH₂R³)+XH→□-W(═O)X(CH₂R¹)(CH₂R²)+R³CH₃
(c) Reaction scheme 2 for obtaining catalysts of formula (II):
□-OH+Mo(═O)X(CH₂R¹)(CH₂R²)(CH₂R³)→□-Mo(═O)X(CH₂R¹)(CH₂R²)
(d) Reaction scheme 2bis for obtaining catalysts of formula (II):
□-OH+Mo(═O)(CH₂R¹)(CH₂R²)(CH₂R³)→□-Mo(═O)(CH₂R¹)(CH₂R²)(CH₂R³)
□-Mo(═O)(CH₂R¹)(CH₂R²)(CH₂R³)+XH→□-Mo(═O)X(CH₂R¹)(CH₂R²)+R³CH₃
(e) Reaction scheme 3 for obtaining catalysts of formula (III):
(□)₂W(═O)Cl₂+Sn(CH₂R¹)₂(CH₂R²)₂→(□)₂W(═O)(CH₂R¹)(CH₂R²)
(f) Reaction scheme 3bis for obtaining catalysts of formula (III):
□-OH+□-OH+W(═O)(CH₂R¹)(CH₂R²)(X′₂)→(□)₂W(═O)(CH₂R¹)(CH₂R²)+2X′H
(g) Reaction scheme 4 for obtaining catalysts of formula (IV):
(□)₂MO(═O)Cl₂+Sn(CH₂R¹)₂(CH₂R²)→(□)₂Mo(═O)(CH₂R¹)(CH₂R²)
(h) Reaction scheme 4bis for obtaining catalysts of formula (IV):
□-OH+□-OH+Mo(═O)(CH₂R¹)(CH₂R²)(X′)₂→(□)₂Mo(═O)(CH₂R¹)(CH₂R²)+2X′H
(i) Reaction scheme 6 for obtaining catalysts of formula (VI):
□-OH+□-OH+Mo(═O)(═CHR¹)(X″)₂→(□)₂Mo(═O)(═CHR¹)+2X′H
(j) Reaction scheme 7 for obtaining catalysts of formula (VII):
□-OH+Mo(═NR4)(═CHR⁵)(G)₂→(□)Mo(═NR4)G(═CHR⁵)+GH
(k) Reaction scheme 8 for obtaining catalysts of formula (VIII):
□-OL^{k}-OH+Mo(═NR⁴)(═CHR⁵)(G)₂→(□-OL^{k}O)Mo(═NR⁴)G(═CHR⁵)+GH
wherein
R¹ and R², are independently to each other, selected from hydrogen, linear or branched alkyl groups, the alkyl group preferably having from 1 to 12 carbon atoms, —C(CH₃)₃, -Phenyl, —Si(CH₃)₃, —C(CH₃)₂Ph, preferably R¹ and R², are independently to each other, selected from —H, methyl, ethyl, propyl, isopropyl, butyl, isobutyl, pentyl, isopentyl, n-hexyl, —C(CH₃)₃, -Phenyl, —Si(CH₃)₃, —C(CH₃)₂Ph,
being understood that R¹ and R² cannot be both hydrogen in formula (III),
R⁴ represents a radical selected from aliphatic and aromatic hydrocarbyl radicals, optionally comprising one or more heteroatoms, preferably comprising from 1 to 36 carbon atoms, preferably from 2 to 28 carbon atoms, more preferably from 3 to 24 carbon atoms,
R⁵ is selected from hydrogen, linear or branched alkyl groups, —C(CH₃)₃, -Phenyl Ph), —Si(CH₃)₃, or —C(CH₃)₂Ph,
G is selected from alkoxy groups, aryloxy groups, siloxy groups or pyrolidyl groups,
L^{k} represents a divalent linker, preferably chosen from a linear, branched or cyclic alkylene, having preferably from 1 to 12 carbon atoms, or an arylene group optionally substituted having preferably from 6 to 12 carbon atoms,
R³ is selected from hydrogen, linear or branched alkyl groups, the alkyl group preferably having from 1 to 12 carbon atoms, —C(CH₃)₃, -Phenyl, —Si(CH₃)₃], —C(CH₃)₂Ph, preferably R³ is selected from —H, methyl, ethyl, propyl, isopropyl, butyl, isobutyl, pentyl, isopentyl, n-hexyl, —C(CH₃)₃, -Phenyl, —Si(CH₃)₃, —C(CH₃)₂Ph,
X′ and X″ are independently to each other selected from chlorine, bromine, fluorine, aryloxy groups, siloxy groups or pyrolidyl groups, preferably X′ and X″ are selected from chlorine, bromine, fluorine or one of the following groups: 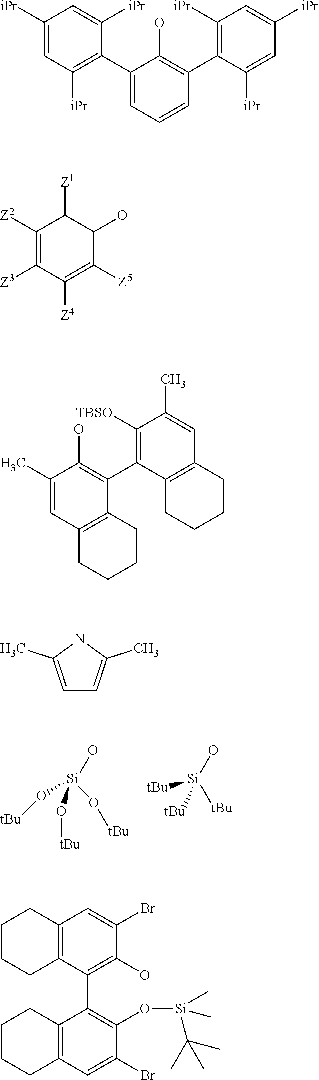
with Z¹, Z², Z³, Z⁴ and Z⁵ are independently to each other selected from hydrogen, methyl, tertio-butyl, adamantyl, mesityl, trifluoromethyl, fluoro, preferably Z²═Z³═Z⁴═H and Z¹ is identical to Z⁵ and is selected from methyl, tertio-butyl, adamantyl, mesityl.

23. The method for the production of poly-alpha-olefins (PAO), said method comprising:
a) producing alpha-olefins, more particularly C₁₀ alpha-olefins, according to the process of claim 1;
b) oligomerizing the alpha-olefins produced in step a); and
c) optionally hydrogenating the oligomer produced in step b).

24. The method according to claim 23, wherein the poly-alpha-olefins are C₃₀ poly-alpha-olefins, wherein step i) comprises the production of C₁₀ alpha-olefins, preferably 1-decene, and wherein the oligomerization reaction in step ii) is a trimerization reaction.
